(19) United States
(12) Patent Application Publication
(10) Pub. No.: US 2023/0398334 A1
(43) Pub. Date: Dec. 14, 2023

(54) COATED BALLOON CATHETER FOR TREATING ARTERIOVENOUS FISTULA AND METHODS OF USING SAME

(71) Applicant: Alucent Biomedical, Inc., Salt Lake City, UT (US)

(72) Inventors: RB Eugene HAYES, Salt Lake City, UT (US); Krishna ROCHA-SINGH, Springfield, IL (US); Venita CHANDRA, Hillsborough, CA (US)

(21) Appl. No.: 17/837,830

(22) Filed: Jun. 10, 2022

(51) Int. Cl. A61M 25/10; A61M 1/36; A61M 25/01

(52) U.S. Cl. CPC A61M 25/1011; A61M 1/3655; A61M 25/0108; A61M 2025/105; A61M 2025/1093; A61M 2025/1086; A61M 2025/1079; A61M 2025/1015; A61M 2025/1075

(57) ABSTRACT

An apparatus includes a catheter shaft extending from a proximal end to a distal tip. The apparatus includes a distal balloon positioned on a distal segment of the catheter shaft proximal to the distal tip. The distal balloon is coated with a Natural Vascular Scaffolding (NVS) treatment. The NVS treatment is configured to enhance maturation of an arteriovenous fistula.

### BACKGROUND

### Technical Field

Systems and methods consistent with the present invention generally relate to apparatus and methods to treat an arteriovenous fistula (AVF). More particularly, systems and methods consistent with the invention relate to the catheters, and catheter systems to deliver drugs to an AVF and promote AVF maturation.

### Discussion of the Related Art

End stage renal disease (ESRD) is characterized by complete or near complete failure of kidney function. For patients with ESRD, hemodialysis is a commonly used therapy in which the patient's blood is removed, filtered through a machine, and returned to the body. Vascular access for hemodialysis may be provided, for example, by an AVF.

An AVF is created by surgically connecting an artery to a vein. Once created, the high blood pressure of the artery causes increased blood flow to the vein. The forces of pulsatile pressure initiate a remodeling response referred to as maturation. An AVF must mature before it can tolerate routine cannulation for dialysis. Successful maturation requires that the venous vessel walls thicken by outward remodeling, meaning that the lumen size remains substantially unchanged.

The forces of pulsatile pressure associated with AVF creation, however, may damage the extracellular matrix at the luminal wall. This can increase proliferative signals, which can lead to inward stenotic processes that decrease lumen size. AVF maturation failure is caused by luminal stenosis due to excessive neointimal hyperplasia and/or impaired outward remodeling. AVF maturation is unsuccessful not only if the vessel wall does not thicken, but also if vessel wall thickening is accompanied by a decrease in lumen size. If maturation fails, the surgical procedure must be repeated.

Because it takes time for maturation to occur and ERSD patients are experiencing complete or near complete failure of kidney function, it is desirable to minimize the time between canulization and AVF creation while ensuring the success of the AVF.

### SUMMARY

An apparatus according to exemplary embodiments of the disclosure includes a catheter shaft extending from a proximal end to a distal tip; and a distal balloon positioned on a distal segment of the catheter shaft proximal to the distal tip, the distal balloon coated with a Natural Vascular Scaffolding (NVS) treatment; wherein the NVS treatment is configured to enhance maturation of an arteriovenous fistula.

An exemplary method according to the present disclosure for maturing an arteriovenous fistula includes providing a catheter into a blood vessel, the catheter including: a catheter shaft extending from a proximal end to a distal tip; and a distal balloon positioned on a distal segment of the catheter shaft proximal to the distal tip, the distal balloon coated with a Natural Vascular Scaffolding (NVS) treatment; advancing the catheter so that the distal balloon is disposed within the arteriovenous fistula; expanding the distal balloon. In certain embodiments, the method further comprises transmitting light down a light fiber arranged within or along the catheter shaft in order to activate the NVS treatment.

### BRIEF DESCRIPTION OF THE DRAWINGS

The accompanying drawings, which are incorporated in and constitute a part of this disclosure, illustrate various embodiments and aspects of the present disclosure. In the drawings:

FIG. 1 is a side elevational view of an exemplary apparatus including a catheter, according to embodiments of the present disclosure;

FIG. 2 is a side elevational view of the exemplary catheter with an olive shaped distal tip;

FIG. 3A is a side elevational view of an alternatively shaped distal tip;

FIG. 3B is a side elevational view of an alternatively shaped distal tip;

FIG. 3C is a side elevational view of an alternatively shaped distal tip;

FIG. 3D is a side elevational view of an alternatively shaped distal tip;

FIG. 3E is a side elevational view of an alternatively shaped distal tip;

FIG. 3F is a side elevational view of an alternatively shaped distal tip;

FIG. 3G is a side elevational view of an alternatively shaped distal tip;

FIG. 4A is a perspective view of the exemplary catheter including a protrusion;

FIG. 4B is a perspective view of the exemplary catheter including a coated shoulder;

FIG. 5 is a perspective partial section view of the exemplary catheter of FIG. 1;

FIG. 6 is a detailed section view of a distal portion of the catheter of FIG. 1;

FIG. 7A is a side elevational view of a proximal portion of the catheter, consistent with embodiments of the present disclosure;

FIG. 7B is a side elevational view of another embodiment of the proximal portion of the catheter, consistent with embodiments of the present disclosure;

FIG. 8 is a cross-sectional view taken along line 5-5 of FIG. 1;

FIG. 9A is a side elevational view of a coated balloon of the catheter with a marker band having echogenic drill holes;

FIG. 9B is a side elevational view of the coated balloon of the catheter with a marker band having an echogenic roughened surface;

FIG. 10A is a side elevational view of a distal tip having an echogenic roughened surface;

FIG. 10B is a side elevational view of a distal tip having an echogenic rod or tube disposed therein;

FIG. 11A is a cross-sectional view of the distal end of an alternative embodiment of a catheter;

FIG. 11B is a cross-sectional view of the distal end of an alternative embodiment of a catheter;

FIG. 11C is a cross-sectional view of the distal end of an alternative embodiment of a catheter;

FIG. 12 is a side elevational view of an exemplary apparatus including a catheter, according to embodiments of the present disclosure;

FIG. 13 is a cross-sectional view taken along line 8-8 of FIG. 12;

FIG. 14A is a cross-sectional view of the distal end of an alternative embodiment of a catheter;

FIG. 14B is a cross-sectional view of the distal end of an alternative embodiment of a catheter;

FIG. 15 is a detailed section view of a distal portion of an exemplary apparatus including a catheter, according to embodiments of the present disclosure;

FIG. 16 is a cross-sectional view of the catheter of FIG. 10; and

FIG. 17 is a flowchart of a method for using a catheter.

### DETAILED DESCRIPTION

The following detailed description refers to the accompanying drawings. Wherever possible, the same reference numbers are used in the drawings and in the following description to refer to the same or similar parts. While several exemplary embodiments and features of the invention are described herein, modifications, adaptations, and other implementations are possible without departing from the spirit and scope of the disclosure. For example, substitutions, additions, or modifications may be made to the components illustrated in the drawings, and the exemplary methods described herein may be modified by substituting, reordering, or adding steps to the disclosed methods. Accordingly, the following detailed description does not limit the disclosure. Instead, the proper scope of the disclosure is defined by the appended claims.

As used herein, the following definitions shall apply unless otherwise indicated.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “maturation” of an AVF refers to an increase in AVF open lumen area without significantly affecting the area of neointimal hyperplasia. A “mature” AVF has sufficient open lumen area (i.e. outward remodeling) and limited neointimal hyperplasia (i.e. inward remodeling) to allow increases in AVF blood flow for dialysis. In some embodiments, the increase in a mature AVF open lumen area occurs without significantly decreasing the compliance of the vessel.

As used herein “Natural Vascular Scaffolding” or “NVS” treatment or therapy refers to treatment of a substrate using at least one active agent, such as at least one active agent chosen from the Compound of Formula (I) and pharmaceutically acceptable salts thereof according to the present disclosure. NVS treatment interlinks collagen and elastin by covalently linking these proteins via photoactivation. Similarly, as used herein, a “Natural Vascular Scaffolding” or “NVS” solution refers to a solution comprising at least one active agent, such as at least one active agent chosen from the Compound of Formula (I) and pharmaceutically acceptable salts thereof according to the present disclosure.

As used herein, “phosphate buffered saline” or “PBS” treatment or solution refers to treatment with or solution of phosphate buffered saline that, unless indicated, does not comprise at least one active agent as defined herein. As used herein, a substrate or subject treated with “phosphate buffered saline” or “PBS” would be in a “no-NVS” group.

The “at least one active agent” is chosen from dimeric naphthalimide compounds. Certain dimeric naphthalimide compounds have been previously disclosed. See, e.g., U.S. Pat. No. 6,410,505 B2. For example, a dimeric naphthalimide compound, 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione), also known as 10-8-10 dimer, 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbis(oxy-2,1-ethanediyl)]bis[6-({2-[2-(2-aminoethoxy)ethoxy]ethyl}amino)-1H-benzo[de]isoquinoline-1,3(2H)-dione]; and 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9Cl), and herein referred to as Compound of Formula (I), has been disclosed. Id.

In some embodiments, the at least one active agent is chosen from the Compound for Formula (I) and pharmaceutically acceptable salts thereof. The “Compound of Formula (I)” as used herein may be described by the structure:

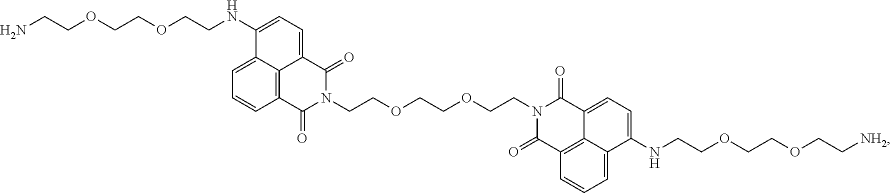 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

by the chemical names 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione); 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbis(oxy-2,1-ethanediyl)]bis[6-({2-[2-(2-aminoethoxy)ethoxy]-ethyl}amino)-1H-benzo[de]isoquinoline-1,3(2H)-dione]; or 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9Cl), or by the Chemical Abstract Services (CAS) Registry No. 438200-66-9.

As used herein, “Compound of Formula (I)” includes one or more of the conformational forms of the compound. Unless stated otherwise, compounds depicted herein coexisting with tautomeric forms are within the scope of the disclosure. Additionally, unless stated otherwise, structures depicted herein are also meant to include compounds that differ only in the presence of one or more isotopically enriched atoms. For example, compounds having the depicted structures except for the replacement of hydrogen by deuterium or tritium, or the replacement of a carbon atom by ¹³C- or ¹⁴C-enriched carbon atom are within the scope of this disclosure.

The at Least One Active Agent

In some embodiments, the at least one active agent is chosen from dimeric naphthalimides and pharmaceutically acceptable salts thereof, e.g., the dimeric naphthalimides disclosed in U.S. Pat. No. 6,410,505 B2. In some embodiments, the at least one active agent is a Compound of Formula (I). In some embodiments, the at least one active agent is chosen from a Compound of Formula (I) and pharmaceutically acceptable salts thereof.

In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate. In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

Compositions

In some embodiments, the methods provided herein comprise administration of a composition comprising at least one active agent to an AVF. In some embodiments, the at least one active agent is present in an amount ranging from 0.01% to 5% by weight of the composition. In some embodiments, the at least one active agent is present in an amount ranging from 0.01% to 4% by weight of the composition. In some embodiments, the at least one active agent is present in an amount ranging from 0.01% to 2.5% by weight of the composition. In some embodiments, the at least one active agent is present in an amount of 0.01%, 0.03%, 0.05%, 0.07%, 0.09%, 0.11%, 0.13%, 0.15%, 0.17%, 0.19%, 0.21%, 0.23%, 0.25%, 0.27%, 0.29%, 0.31%, 0.33%, 0.35%, 0.37%, 0.39%, 0.41%, 0.43%, 0.45%, 0.47%, 0.49%, 0.51%, 0.53%, 0.55%, 0.57%, 0.59%, 0.61%, 0.63%, 0.65%, 0.67%, 0.69%, 0.71%, 0.73%, 0.75%, 0.77%, 0.79%, 0.81%, 0.83%, 0.85%, 0.87%, 0.89%, 0.91%, 0.93%, 0.95%, 0.97%, 0.99%, 1.01%, 1.03%, 1.05%, 1.07%, 1.09%, 1.11%, 1.13%, 1.15%, 1.17%, 1.19%, 1.21%, 1.23%, 1.25%, 1.27%, 1.29%, 1.31%, 1.33%, 1.35%, 1.37%, 1.39%, 1.41%, 1.43%, 1.45%, 1.47%, 1.49%, 1.51%, 1.53%, 1.55%, 1.57%, 1.59%, 1.61%, 1.63%, 1.65%, 1.67%, 1.69%, 1.71%, 1.73%, 1.75%, 1.77%, 1.79%, 1.81%, 1.83%, 1.85%, 1.87%, 1.89%, 1.91%, 1.93%, 1.95%, 1.97%, 1.99%, 2.01%, 2.03%, 2.05%, 2.07%, 2.09%, 2.11%, 2.13%, 2.15%, 2.17%, 2.19%, 2.21%, 2.23%, 2.25%, 2.27%, 2.29%, 2.31%, 2.33%, 2.35%, 2.37%, 2.39%, 2.41%, 2.43%, 2.45%, 2.47%, or 2.49% by weight of the composition. In some embodiments, the at least one active agent is present in an amount of 0.1%, 0.2%, 0.3%, 0.4%, 0.5%, 0.6%, 0.7%, 0.8%, 0.9%, 1%, 1.1%, or 1.2% by weight of the composition.

In some embodiments, the at least one active agent is present in an amount ranging from 0.01% to 1% by weight of the composition. In some embodiments, the at least one active agent is present in an amount ranging from 0.3% to 0.6% by weight of the composition. In some embodiments, the at least one active agent is present in an amount ranging from 0.4% to 0.5% by weight of the composition. In some embodiments, the at least one active agent is present in an amount ranging from 0.1% to 0.5% by weight of the composition. In some embodiments, the at least one active agent is present in an amount of 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, or 0.5% by weight of the composition. In some embodiments, the at least one active agent is present in an amount ranging from 0.1% to 0.3% by weight of the composition.

In some embodiments, the at least one active agent is present in an amount of 0.01%, 0.02%, 0.03%, 0.04%, 0.05%, 0.06%, 0.07%, 0.08%, 0.09%, 0.1%, 0.12%, 0.14%, 0.16%, 0.18%, 0.2%, 0.22%, 0.24%, 0.26%, 0.28%, 0.3%, 0.32%, 0.34%, 0.36%, 0.38%, 0.4%, 0.42%, 0.44%, 0.48%, 0.5%, 0.52%, 0.54%, 0.56%, 0.58%, 0.6%, 0.62%, 0.64%, 0.66%, 0.68%, 0.7%, 0.72%, 0.74%, 0.76%, 0.78%, 0.8%, 0.82%, 0.84%, 0.86%, 0.88%, 0.9%, 0.92%, 0.94%, 0.96%, 0.98%, or 1% by weight of the composition.

In some embodiments, the at least one active agent is present in an amount of 0.08% by weight of the composition. In some embodiments, the at least one active agent is present in an amount of 0.1% by weight of the composition. In some embodiments, the at least one active agent is present in an amount of 0.12% by weight of the composition. In some embodiments, the at least one active agent is present in an amount of 0.14% by weight of the composition. In some embodiments, the at least one active agent is present in an amount of 0.16% by weight of the composition. In some embodiments, the at least one active agent is present in an amount of 0.18% by weight of the composition. In some embodiments, the at least one active agent is present in an amount of 0.2% by weight of the composition. In some embodiments, the at least one active agent is present in an amount of 0.22% by weight of the composition. In some embodiments, the at least one active agent is present in an amount of 0.24% by weight of the composition. In some embodiments, the at least one active agent is present in an amount of 0.26% by weight of the composition. In some embodiments, the at least one active agent is present in an amount of 0.36% by weight of the composition. In some embodiments, the at least one active agent is present in an amount of 0.38% by weight of the composition. In some embodiments, the at least one active agent is present in an amount of 0.4% by weight of the composition. In some embodiments, the at least one active agent is present in an amount of 0.42% by weight of the composition. In some embodiments, the at least one active agent is present in an amount of 0.44% by weight of the composition. In some embodiments, the at least one active agent is present in an amount of 0.46% by weight of the composition. In some embodiments, the at least one active agent is present in an amount of 0.48% by weight of the composition. In some embodiments, the at least one active agent is present in an amount of 0.5% by weight of the composition.

At Least One Solvent

In some embodiments, the composition further comprises at least one solvent. In some embodiments, the at least one solvent is chosen from water, ethanol, isopropanol, polyethylene glycols, and propylene glycols.

In some embodiments, the at least one solvent is water. In some embodiments, the at least one solvent is ethanol. In some embodiments, the at least one solvent is isopropanol. In some embodiments, the at least one solvent is chosen from polyethylene glycols. In some embodiments, the at least one solvent is chosen from propylene glycols. In some embodiments, the polyethylene glycols are chosen from polyethylene glycol 300, polyethylene glycol 400, polyethylene glycol 500, and polyethylene glycol 600.

In some embodiments, the at least one solvent is present in an amount of q.s. 100% by weight of the composition.

At Least One Tonicity Agent

In some embodiments, the composition further comprises at least one tonicity agent. Without being bound by any theory, in some embodiments, at least one tonicity agent may be added to modulate the solute concentration of a composition.

In some embodiments, the at least one tonicity agent is chosen from dextrose, sorbitol, lactose, mannitol, sodium chloride, potassium chloride, and glycerol. In some embodiments, the at least one tonicity agent is chosen from sodium chloride and potassium chloride. In some embodiments, the at least one tonicity agent is sodium chloride and potassium chloride.

In some embodiments, the at least one tonicity agent is dextrose. In some embodiments, the at least one tonicity agent is sorbitol. In some embodiments, the at least one tonicity agent is lactose. In some embodiments, the at least one tonicity agent is mannitol. In some embodiments, the at least one tonicity agent is sodium chloride. In some embodiments, the at least one tonicity agent is potassium chloride. In some embodiments, the at least one tonicity agent is glycerol.

In some embodiments, the at least one tonicity agent is present in an amount up to 5% by weight of the composition. In some embodiments, the at least one tonicity agent is present in an amount up to 4% by weight of the composition. In some embodiments, the at least one tonicity agent is present in an amount up to 3% by weight of the composition. In some embodiments, the at least one tonicity agent is present in an amount of up to 2.5% by weight of the composition. In some embodiments, the at least one tonicity agent is present in an amount of up to 2% by weight of the composition. In some embodiments, the at least one tonicity agent is present in an amount of up to 1.5% by weight of the composition. In some embodiments, the at least one tonicity agent is present in an amount of up to 1% by weight of the composition. In some embodiments, the at least one tonicity agent is present in an amount of up to 0.5% by weight of the composition.

In some embodiments, the at least one tonicity agent is present in an amount ranging from 0.25% to 3% by weight of the composition. In some embodiments, the at least one tonicity agent is present in an amount ranging from 0.25% to 2.5% by weight of the composition. In some embodiments, the at least one tonicity agent is present in an amount ranging from 0.5% to 2% by weight of the composition. In some embodiments, the at least one tonicity agent is present in an amount ranging from 0.5% to 1.5% by weight of the composition.

In some embodiments, the at least one tonicity agent is present in an amount of 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, or 0.9% by weight of the composition. In some embodiments, the at least one tonicity agent is present in an amount of 0.72%, 0.74%, 0.76%, 0.78%, 0.8%, 0.82%, or 0.84% by weight of the composition. In some embodiments, the at least one tonicity agent is present in an amount of 0.72% by weight of the composition. In some embodiments, the at least one tonicity agent is present in an amount of 0.74% by weight of the composition. In some embodiments, the at least one tonicity agent is present in an amount of 0.76% by weight of the composition. In some embodiments, the at least one tonicity agent is present in an amount of 0.78% by weight of the composition. In some embodiments, the at least one tonicity agent is present in an amount of 0.8% by weight of the composition. In some embodiments, the at least one tonicity agent is present in an amount of 0.82% by weight of the composition. In some embodiments, the at least one tonicity agent is present in an amount of 0.84% by weight of the composition.

At Least One Buffer

In some embodiments, the composition further comprises at least one buffer. Without being bound by any theory, in some embodiments, at least one buffer may be added to maintain a desired pH or pH range.

In some embodiments, the at least one buffer is chosen from a potassium salt, a sodium salt, and maleic acid. In some embodiments, the at least one buffer is a sodium salt. In some embodiments, the at least one buffer is a potassium salt. In some embodiments, the at least one buffer is maleic acid. In some embodiments, the at least one buffer comprises a potassium salt and a sodium salt.

In some embodiments, the potassium salt is chosen from potassium phosphate, potassium citrate, potassium acetate, potassium lactate, and potassium tartrate. In some embodiments, the sodium salt is chosen from sodium phosphate, sodium citrate, sodium acetate, sodium lactate, and sodium tartrate. In some embodiments, the at least one buffer is chosen from potassium phosphate and sodium phosphate. In some embodiments, the at least one buffer comprises potassium phosphate and sodium phosphate.

In some embodiments, the potassium phosphate is chosen from potassium phosphate monobasic, potassium phosphate dibasic, and potassium phosphate tribasic. In some embodiments, the potassium phosphate is chosen from potassium phosphate monobasic and potassium phosphate dibasic. In some embodiments, the potassium phosphate is potassium phosphate monobasic. In some embodiments, the potassium phosphate is potassium phosphate dibasic. In some embodiments, the potassium phosphate is potassium phosphate tribasic.

In some embodiments, the sodium phosphate is chosen from sodium phosphate monobasic, sodium phosphate dibasic, and sodium phosphate tribasic. In some embodiments, the sodium phosphate is chosen from sodium phosphate monobasic and sodium phosphate dibasic. In some embodiments, the sodium phosphate is sodium phosphate monobasic. In some embodiments, the sodium phosphate is sodium phosphate dibasic. In some embodiments, the sodium phosphate is sodium phosphate tribasic.

In some embodiments, the at least one buffer is chosen from sodium phosphate dibasic and potassium phosphate monobasic. In some embodiments, the at least one buffer is sodium phosphate dibasic and potassium phosphate monobasic.

In some embodiments, the at least one buffer is anhydrous.

In some embodiments, the at least one buffer is present in an amount of up to 2.5% by weight of the composition. In some embodiments, the at least one buffer is present in an amount of up to 2% by weight of the composition. In some embodiments, the at least one buffer is present in an amount of up to 1.5% by weight of the composition. In some embodiments, the at least one buffer is present in an amount of up to 1% by weight of the composition. In some embodiments, the at least one buffer is present in an amount of up to 0.5% by weight of the composition.

In some embodiments, the at least one buffer is present in an amount ranging from 0.05% to 0.4% by weight of the composition. In some embodiments, the at least one buffer is present in an amount of 0.06%, 0.08%, 0.1%, 0.12%, 0.14%, 0.16%, 0.18% 0.2%, 0.22%, 0.24%, 0.26%, 0.28%, or 0.3% by weight of the composition. In some embodiments, the at least one buffer is present in an amount ranging from 0.1% to 0.2% by weight of the composition. In some embodiments, the at least one buffer is present in an amount ranging from 0.08% to 0.16% by weight of the composition. In some embodiments, the at least one buffer is present in an amount of 0.1% by weight of the composition. In some embodiments, the at least one buffer is present in an amount of 0.11% by weight of the composition. In some embodiments, the at least one buffer is present in an amount of 0.12% by weight of the composition. In some embodiments, the at least one buffer is present in an amount of 0.13% by weight of the composition. In some embodiments, the at least one buffer is present in an amount of 0.14% by weight of the composition. In some embodiments, the at least one buffer is present in an amount of 0.15% by weight of the composition. In some embodiments, the at least one buffer is present in an amount of 0.16% by weight of the composition. In some embodiments, the at least one buffer is present in an amount of 0.17% by weight of the composition. In some embodiments, the at least one buffer is present in an amount of 0.18% by weight of the composition. In some embodiments, the at least one buffer is present in an amount of 0.19% by weight of the composition. In some embodiments, the at least one buffer is present in an amount of 0.2% by weight of the composition.

In some embodiments, the at least one buffer comprises sodium phosphate in an amount ranging from 0.01% to 0.03% by weight of the composition and potassium phosphate in an amount ranging from 0.1% to 0.14% by weight of the composition.

At Least One pH Modulation Agent

In some embodiments, the composition further comprises at least one pH modulation agent. Without being bound by any theory, in some embodiments, at least one pH modulation agent may be added to achieve a desired pH of the composition.

In some embodiments, the at least one pH modulation agent is chosen from acetic acid, carbonic acid, citric acid, sodium bicarbonate, and sodium hydroxide. In some embodiments, the at least one pH modulation agent is acetic acid. In some embodiments, the at least one pH modulation agent is carbonic acid. In some embodiments, the at least one pH modulation agent is citric acid. In some embodiments, the at least one pH modulation agent is sodium bicarbonate. In some embodiments, the at least one pH modulation agent is sodium hydroxide.

In some embodiments, the at least one pH modulation agent is chosen from acetic acid and sodium hydroxide. In some embodiments, the at least one pH modulation agent is acetic acid and sodium hydroxide.

In some embodiments, the at least one pH modulation agent is present in an amount of q.s. to desired pH.

At Least One Viscosity Agent

In some embodiments, the composition further comprises at least one viscosity agent. Without being bound by any theory, in some embodiments, at least one viscosity agent may be added to achieve a desired viscosity or thickness of the composition.

In some embodiments, the at least one viscosity agent is chosen from gelatin, methylcellulose, hydroxypropyl cellulose, hydroxypropyl methylcellulose, hydroxyethyl cellulose, hydroxypropylmethyl cellulose, methylhydroxyethyl cellulose, methylhydroxypropyl cellulose, hydroxyethylcarboxymethyl cellulose, carboxymethyl cellulose, carboxymethylhydroxyethyl cellulose, and sodium carboxymethylcellulose. In some embodiments, the at least one viscosity agent is chosen from methylcellulose, sodium carboxymethylcellulose, and hydroxypropyl cellulose.

In some embodiments, the at least one viscosity agent is gelatin. In some embodiments, the at least one viscosity agent is methylcellulose. In some embodiments, the at least one viscosity agent is hydroxypropyl cellulose. In some embodiments, the at least one viscosity agent is hydroxypropyl methylcellulose. In some embodiments, the at least one viscosity agent is hydroxyethyl cellulose. In some embodiments, the at least one viscosity agent is hydroxypropylmethyl cellulose. In some embodiments, the at least one viscosity agent is methylhydroxyethyl cellulose. In some embodiments, the at least one viscosity agent is methylhydroxypropyl cellulose. In some embodiments, the at least one viscosity agent is hydroxyethylcarboxymethyl cellulose. In some embodiments, the at least one viscosity agent is carboxymethyl cellulose. In some embodiments, the at least one viscosity agent is carboxymethylhydroxyethyl cellulose. In some embodiments, the at least one viscosity agent is sodium carboxymethylcellulose.

In some embodiments, the at least one viscosity agent is present in an amount of 2.5% or less by weight of the composition. In some embodiments, the at least one viscosity agent is present in an amount of 2% or less by weight of the composition. In some embodiments, the at least one viscosity agent is present in an amount of 1.5% or less by weight of the composition. In some embodiments, the at least one viscosity agent is present in an amount of 1% or less by weight of the composition. In some embodiments, the at least one viscosity agent is present in an amount of 0.5% or less by weight of the composition.

In some embodiments, the at least one viscosity agent is present in an amount ranging from 0.05% to 1% by weight of the composition. In some embodiments, the at least one viscosity agent is present in an amount ranging from 0.1% to 0.5% by weight of the composition. In some embodiments, the at least one viscosity agent is present in an amount ranging from 0.1% to 0.3% by weight of the composition. In some embodiments, the at least one viscosity agent is present in an amount of 0.15%, 0.16%, 0.17%, 0.18%, 0.19%, 0.2%, 0.21%, 0.22%, 0.23%, 0.24%, or 0.25% by weight of the composition.

In some embodiments, the at least one viscosity agent is present in an amount of 0.15% by weight of the composition. In some embodiments, the at least one viscosity agent is present in an amount of 0.16% by weight of the composition. In some embodiments, the at least one viscosity agent is present in an amount of 0.17% by weight of the composition. In some embodiments, the at least one viscosity agent is present in an amount of 0.18% by weight of the composition. In some embodiments, the at least one viscosity agent is present in an amount of 0.19% by weight of the composition. In some embodiments, the at least one viscosity agent is present in an amount of 0.2% by weight of the composition. In some embodiments, the at least one viscosity agent is present in an amount of 0.21% by weight of the composition. In some embodiments, the at least one viscosity agent is present in an amount of 0.22% by weight of the composition. In some embodiments, the at least one viscosity agent is present in an amount of 0.23% by weight of the composition. In some embodiments, the at least one viscosity agent is present in an amount of 0.24% by weight of the composition. In some embodiments, the at least one viscosity agent is present in an amount of 0.25% by weight of the composition.

At Least One Penetration Enhancer

In some embodiments, the composition further comprises at least one penetration enhancer. Without being bound by any theory, in some embodiments, at least one penetration enhancer may be added to increase the amount of the at least one active agent delivered to the desired location.

In some embodiments, the at least one penetration enhancer is chosen from benzyl alcohol, diethylene glycol monoethyl ether, caprylic acid, and sodium oleate. In some embodiments, the at least one penetration enhancer is benzyl alcohol. In some embodiments, the at least one penetration enhancer is diethylene glycol monoethyl ether. In some embodiments, the at least one penetration enhancer is caprylic acid. In some embodiments, the at least one penetration enhancer is sodium oleate.

In some embodiments, the at least one penetration enhancer is present in an amount ranging from 0.01% to 1% by weight of the composition. In some embodiments, the at least one penetration enhancer is present in an amount ranging from 0.01% to 0.5% by weight of the composition. In some embodiments, the at least one penetration enhancer is present in an amount ranging from 0.05% to 0.25% by weight of the composition. In some embodiments, the at least one penetration enhancer is present in an amount of 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, or 1% by weight of the composition.

At Least One Stabilizing Agent

In some embodiments, the composition further comprises at least one stabilizing agent. Without being bound by any theory, in some embodiments, at least one stabilizing agent may be added to retard or completely prevent degradation of the at least one active agent and/or the stabilizing agent may retard or completely prevent the appearance of impurities in the composition.

In some embodiments, the at least one stabilizing agent is chosen from ascorbic acid, butylated hydroxytoluene, citric acid, benzoic acid, and sodium metabisulfite. In some embodiments, the at least one stabilizing agent is ascorbic acid. In some embodiments, the at least one stabilizing agent is butylated hydroxytoluene. In some embodiments, the at least one stabilizing agent is citric acid. In some embodiments, the at least one stabilizing agent is benzoic acid. In some embodiments, the at least one stabilizing agent is sodium metabisulfite.

In some embodiments, the at least one stabilizing agent is present in an amount ranging from 0.005% to 1% by weight of the composition. In some embodiments, the at least one stabilizing agent is present in an amount ranging from 0.005% to 0.25%, 0.5%, 0.75%, or 1% by weight of the composition.

In some embodiments, the at least one stabilizing agent is present in an amount ranging from 0.01% to 1% by weight of the composition. In some embodiments, the at least one stabilizing agent is present in an amount ranging from 0.01% to 0.25%, 0.5%, 0.75%, or 1% by weight of the composition

In some embodiments, the at least one stabilizing agent is present in an amount ranging from 0.1% to 1% by weight of the composition. In some embodiments, the at least one stabilizing agent is present in an amount ranging from 0.1% to 0.25%, 0.5%, 0.75%, or 1% by weight of the composition.

In some embodiments, the at least one stabilizing agent is present in an amount of 0.005%, 0.01%, 0.025%, 0.05%, 0.1%, 0.15%, 0.2%, 0.25%, 0.3%, 0.4%, 0.5%, 0.75%, or 1% by weight of the composition. In some embodiments, the at least one stabilizing agent is present in an amount of 0.005% by weight of the composition. In some embodiments, the at least one stabilizing agent is present in an amount of 0.01% by weight of the composition. In some embodiments, the at least one stabilizing agent is present in an amount of 0.025% by weight of the composition. In some embodiments, the at least one stabilizing agent is present in an amount of 0.05% by weight of the composition. In some embodiments, the at least one stabilizing agent is present in an amount of 0.1% by weight of the composition. In some embodiments, the at least one stabilizing agent is present in an amount of 0.15% by weight of the composition. In some embodiments, the at least one stabilizing agent is present in an amount of 0.2% by weight of the composition. In some embodiments, the at least one stabilizing agent is present in an amount of 0.25% by weight of the composition. In some embodiments, the at least one stabilizing agent is present in an amount of 0.3% by weight of the composition. In some embodiments, the at least one stabilizing agent is present in an amount of 0.4% by weight of the composition. In some embodiments, the at least one stabilizing agent is present in an amount of 0.5% by weight of the composition. In some embodiments, the at least one stabilizing agent is present in an amount of 0.75% by weight of the composition. In some embodiments, the at least one stabilizing agent is present in an amount of 1% by weight of the composition.

At Least One Solubilizing Agent

In some embodiments, the composition further comprises at least one solubilizing agent. Without being bound by any theory, in some embodiments, at least one solubilizing agent may be added to increase the solubility of the at least one active agent in a vehicle, e.g., water, by forming, e.g., an emulsion.

In some embodiments, the at least one solubilizing agent is chosen from tocopherols, fixed oils, soybean oil, PEG-15 hydroxystearates, polysorbate 20, polysorbate 80, 2-hydroxypropyl-p-cyclodextrin, and γ-cyclodextrin. In some embodiments, the solubilizing agent is chosen from tocopherols. In some embodiments, the solubilizing agent is chosen from fixed oils. In some embodiments, the solubilizing agent is chosen from PEG-15 hydroxystearates. In some embodiments, the solubilizing agent is polysorbate 20. In some embodiments, the solubilizing agent is polysorbate 80. In some embodiments, the solubilizing agent is 2-hydroxypropyl-3-cyclodextrin. In some embodiments, the solubilizing agent is γ-cyclodextrin.

In some embodiments, fixed oils are chosen from corn oil, cottonseed oil, peanut oil, and sesame oil.

In some embodiments, the at least one solubilizing agent is present in an amount ranging from 0.005% to 10% by weight of the composition. In some embodiments, the at least one solubilizing agent is present in an amount ranging from 0.005% to 0.1%, 0.25%, 0.5%, 1%, 1.5%, 2.5%, 5%, 7.5%, or 10% by weight of the composition. In some embodiments, the at least one solubilizing agent is present in an amount ranging from 0.1% to 10% by weight of the composition. In some embodiments, the at least one solubilizing agent is present in an amount ranging from 0.1% to 0.25%, 0.5%, 1%, 1.5%, 2.5%, 5%, 7.5%, or 10% by weight of the composition.

In some embodiments, the at least one solubilizing agent is present in an amount ranging from 1% to 10% by weight of the composition. In some embodiments, the at least one solubilizing agent is present in an amount ranging from 1% to 1.5%, 2.5%, 5%, 7.5%, or 10% by weight of the composition.

In some embodiments, the at least one solubilizing agent is present in an amount of 0.005%, 0.01%, 0.025%, 0.05%, 0.1%, 0.15%, 0.2%, 0.25%, 0.3%, 0.4%, 0.5%, 0.75%, 1%, 1.5%, 2%, 2.5%, 5%, 7.5%, or 10% by weight of the composition. In some embodiments, the at least one solubilizing agent is present in an amount of 0.005% by weight of the composition. In some embodiments, the at least one solubilizing agent is present in an amount of 0.01% by weight of the composition. In some embodiments, the at least one solubilizing agent is present in an amount of 0.025% by weight of the composition. In some embodiments, the at least one solubilizing agent is present in an amount of 0.05% by weight of the composition. In some embodiments, the at least one solubilizing agent is present in an amount of 0.1% by weight of the composition. In some embodiments, the at least one solubilizing agent is present in an amount of 0.15% by weight of the composition. In some embodiments, the at least one solubilizing agent is present in an amount of 0.2% by weight of the composition. In some embodiments, the at least one solubilizing agent is present in an amount of 0.25% by weight of the composition. In some embodiments, the at least one solubilizing agent is present in an amount of 0.3% by weight of the composition. In some embodiments, the at least one solubilizing agent is present in an amount of 0.4% by weight of the composition. In some embodiments, the at least one solubilizing agent is present in an amount of 0.5% by weight of the composition. In some embodiments, the at least one solubilizing agent is present in an amount of 0.75% by weight of the composition. In some embodiments, the at least one solubilizing agent is present in an amount of 1% by weight of the composition. In some embodiments, the at least one solubilizing agent is present in an amount of 1.5% by weight of the composition. In some embodiments, the at least one solubilizing agent is present in an amount of 2% by weight of the composition. In some embodiments, the at least one solubilizing agent is present in an amount of 2.5% by weight of the composition. In some embodiments, the at least one solubilizing agent is present in an amount of 5% by weight of the composition. In some embodiments, the at least one solubilizing agent is present in an amount of 7.5% by weight of the composition. In some embodiments, the at least one solubilizing agent is present in an amount of 10% by weight of the composition.

At Least One Encapsulation Agent

In some embodiments, the composition further comprises at least one encapsulation agent. Without being bound by any theory, in some embodiments, at least one encapsulation agent may be added to improve delivery, stability, and/or solubility of the at least one active agent by, e.g., encapsulating the at least active agent in a liposome or lipid particle.

In some embodiments, the at least one encapsulation agent is chosen from 1,2-dimyristoyl-sn-glycero-phosphocholine, 1,2-distearoyl-sn-glycero-3-(phosphor-rac-(1-glycerol)), 1,2-distearoyl-sn-glycero-3-phosphocholine, cholesterol, DL-dipalmitoylphosphatidylglycerol, sodium N-(carbonyl-methoxypolyethylene glycol 2000)-1,2-distearoyl-sn-glycero-3-phosphoethanolamine, sodium N-(carbonyl-methoxypolyethylene glycol 2000)-distearoyl-glycerophosphoethanolamine, DL-distearoylphosphatidylcholine, egg phospholipids, and hydrogenated soybean lecithin.

In some embodiments, the at least one encapsulation agent is 1,2-dimyristoyl-sn-glycero-phosphocholine. In some embodiments, the at least one encapsulation agent is 1,2-distearoyl-sn-glycero-3-(phosphor-rac-(1-glycerol)). In some embodiments, the at least one encapsulation agent is 1,2-distearoyl-sn-glycero-3-phosphocholine. In some embodiments, the at least one encapsulation agent is cholesterol. In some embodiments, the at least one encapsulation agent is DL-dipalmitoylphosphatidylglyce-rol. In some embodiments, the at least one encapsulation agent is sodium N-(carbonyl-methoxypolyethylene glycol 2000)-1,2-distearoyl-sn-glycero-3-phosphoethanolamine. In some embodiments, the at least one encapsulation agent is sodium N-(carbonyl-methoxypolyethylene glycol 2000)-distearoyl-glycerophosphoethanolamine. In some embodiments, the at least one encapsulation agent is DL-distearoylphosphatidylcholine. In some embodiments, the at least one encapsulation agent is an egg phospholipid. In some embodiments, the at least one encapsulation agent is hydrogenated soybean lecithin.

In some embodiments, the at least one encapsulation agent is present in an amount ranging from 0.005% to 10% by weight of the composition. In some embodiments, the at least one encapsulation agent is present in an amount ranging from 0.005% to 0.1%, 0.25%, 0.5%, 1%, 1.5%, 2.5%, 5%, 7.5%, or 10% by weight of the composition. In some embodiments, the at least one encapsulation agent is present in an amount ranging from 0.1% to 10% by weight of the composition. In some embodiments, the at least one encapsulation agent is present in an amount ranging from 0.1% to 0.25%, 0.5%, 1%, 1.5%, 2.5%, 5%, 7.5%, or 10% by weight of the composition.

In some embodiments, the at least one encapsulation agent is present in an amount ranging from 1% to 10% by weight of the composition. In some embodiments, the at least one encapsulation agent is present in an amount ranging from 1% to 1.5%, 2.5%, 5%, 7.5%, or 10% by weight of the composition.

In some embodiments, the at least one encapsulation agent is present in an amount of 0.005%, 0.01%, 0.025%, 0.05%, 0.1%, 0.15%, 0.2%, 0.25%, 0.3%, 0.4%, 0.5%, 0.75%, 1%, 1.5%, 2%, 2.5%, 5%, 7.5%, or 10% by weight of the composition. In some embodiments, the at least one encapsulation agent is present in an amount of 0.005% by weight of the composition. In some embodiments, the at least one encapsulation agent is present in an amount of 0.01% by weight of the composition. In some embodiments, the at least one encapsulation agent is present in an amount of 0.025% by weight of the composition. In some embodiments, the at least one encapsulation agent is present in an amount of 0.05% by weight of the composition. In some embodiments, the at least one encapsulation agent is present in an amount of 0.1% by weight of the composition. In some embodiments, the at least one encapsulation agent is present in an amount of 0.15% by weight of the composition. In some embodiments, the at least one encapsulation agent is present in an amount of 0.2% by weight of the composition. In some embodiments, the at least one encapsulation agent is present in an amount of 0.25% by weight of the composition. In some embodiments, the at least one encapsulation agent is present in an amount of 0.3% by weight of the composition. In some embodiments, the at least one encapsulation agent is present in an amount of 0.4% by weight of the composition. In some embodiments, the at least one encapsulation agent is present in an amount of 0.5% by weight of the composition. In some embodiments, the at least one encapsulation agent is present in an amount of 0.75% by weight of the composition. In some embodiments, the at least one encapsulation agent is present in an amount of 1% by weight of the composition. In some embodiments, the at least one encapsulation agent is present in an amount of 1.5% by weight of the composition. In some embodiments, the at least one encapsulation agent is present in an amount of 2% by weight of the composition. In some embodiments, the at least one encapsulation agent is present in an amount of 2.5% by weight of the composition. In some embodiments, the at least one encapsulation agent is present in an amount of 5% by weight of the composition. In some embodiments, the at least one encapsulation agent is present in an amount of 7.5% by weight of the composition. In some embodiments, the at least one encapsulation agent is present in an amount of 10% by weight of the composition.

At Least One Imaging Agent

In some embodiments, the composition further comprises at least one imaging agent. In some embodiments, the at least one imaging agent is a radiographic contrast agent. In some embodiments, when at least one imaging agent is present in the composition, the composition further comprising at least one imaging agent is formed by combining a composition comprising at least one imaging agent and a composition comprising at least one active agent. In some embodiments, the composition comprising at least one active agent is a composition described herein.

In some embodiments, when at least one imaging agent is present in the composition, the composition further comprising at least one imaging agent is formed by combining a composition comprising at least one imaging agent and a composition comprising at least one active agent prior to use of the composition comprising at least one active agent and further comprising at least one imaging agent. In some embodiments, when at least one imaging agent is present in the composition, the composition further comprising at least one imaging agent is formed by combining a composition comprising at least one imaging agent and a composition comprising at least one active agent 15 seconds to 24 hours prior to use of the composition comprising at least one active agent and further comprising at least one imaging agent. In some embodiments, when at least one imaging agent is present in the composition, the composition further comprising at least one imaging agent is formed by combining a composition comprising at least one imaging agent and a composition comprising at least one active agent 15 seconds to 6 hours prior to use of the composition comprising at least one active agent and further comprising at least one active agent. In some embodiments, when at least one imaging agent is present in the composition, the composition further comprising at least one imaging agent is formed by combining a composition comprising at least one imaging agent and a composition comprising at least one active agent 15 seconds to 1 hour prior to use of the composition comprising at least one active agent and further comprising at least one active agent. In some embodiments, when at least one imaging agent is present in the composition, the composition further comprising at least one imaging agent is formed by combining a composition comprising at least one imaging agent and a composition comprising at least one active agent less than 2 hours prior to use of the composition comprising at least one active agent and further comprising at least one imaging agent.

In some embodiments, when at least one imaging agent is present in the composition, the ratio of the volume of a composition comprising at least one imaging agent to the volume of a composition comprising at least one active agent ranges from 0.5:1 to 2.5:1 prior to combination. In some embodiments, when at least one imaging agent is present in the composition, the ratio of the volume of a composition comprising at least one imaging agent to the volume of a composition comprising at least one active agent ranges from 0.5:1 to 1.5:1 prior to combination. In some embodiments, when at least one imaging agent is present in the composition, the ratio of the volume of a composition comprising at least one imaging agent to the volume of a composition comprising at least one active agent ranges from 0.75:1 to 1.25:1 prior to combination.

In some embodiments, when at least one imaging agent is present in the composition, the ratio of the volume of a composition comprising at least one imaging agent to the volume of a composition comprising at least one active agent is 0.5:1 prior to combination. In some embodiments, when at least one imaging agent is present in the composition, the ratio of the volume of a composition comprising at least one imaging agent to the volume of a composition comprising at least one active agent is 0.75:1 prior to combination. In some embodiments, when at least one imaging agent is present in the composition, the ratio of the volume of a composition comprising at least one imaging agent to the volume of a composition comprising at least one active agent is 1:1 prior to combination. In some embodiments, when at least one imaging agent is present in the composition, the ratio of the volume of a composition comprising at least one imaging agent to the volume of a composition comprising at least one active agent is 1.25:1 prior to combination. In some embodiments, when at least one imaging agent is present in the composition, the ratio of the volume of a composition comprising at least one imaging agent to the volume of a composition comprising at least one active agent is 1.5:1 prior to combination. In some embodiments, when at least one imaging agent is present in the composition, the ratio of the volume of a composition comprising at least one imaging agent to the volume of a composition comprising at least one active agent is 1.75:1 prior to combination. In some embodiments, when at least one imaging agent is present in the composition, the ratio of the volume of a composition comprising at least one imaging agent to the volume of a composition comprising at least one active agent is 2:1 prior to combination. In some embodiments, when at least one imaging agent is present in the composition, the ratio of the volume of a composition comprising at least one imaging agent to the volume of a composition comprising at least one active agent is 2.25:1 prior to combination. In some embodiments, when at least one imaging agent is present in the composition, the ratio of the volume of a composition comprising at least one imaging agent to the volume of a composition comprising at least one active agent is 2.5:1 prior to combination.

In some embodiments, when at least one imaging agent is present in the composition, the molar ratio of the at least one imaging agent to the at least one active agent ranges from 300:1 to 50:1. In some embodiments, when at least one imaging agent is present in the composition, the molar ratio of the at least one imaging agent to the at least one active agent ranges from 250:1 to 75:1. In some embodiments, when at least one imaging agent is present in the composition, the molar ratio of the at least one imaging agent to the at least one active agent ranges from 200:1 to 100:1. In some embodiments, when at least one imaging agent is present in the composition, the molar ratio of the at least one imaging agent to the at least one active agent ranges from 175:1 to 125:1. In some embodiments, when at least one imaging agent is present in the composition, the molar ratio of the at least one imaging agent to the at least one active agent is 158:1.

At Least One Lewis Acid

In some embodiments, the composition further comprises at least one Lewis acid. Without being bound by any theory, in some embodiments, at least one Lewis acid may be added to retard or completely prevent degradation of the at least one active agent, the Lewis acid may retard or completely prevent the appearance of impurities in the coating composition, and/or the Lewis acid may retard or completely prevent the appearance of impurities in the coating of a coated article.

In some embodiments, the at least one Lewis acid is Na+. In some embodiments, the at least one Lewis acid is in the form of NaCl. In some embodiments, the at least one Lewis acid is in the form of KCl. In some embodiments, the at least one Lewis acid is K⁺, Mg²⁺, or Ca²⁺.

At Least One Antioxidant

In some embodiments, the composition further comprises at least one antioxidant. Without being bound by any theory, in some embodiments, at least one antioxidant may be added to retard or completely prevent degradation of the at least one active agent, the antioxidant may retard or completely prevent the appearance of impurities in the coating composition, and/or the antioxidant may retard or completely prevent the appearance of impurities in the coating of a coated article.

In some embodiments, the at least one antioxidant is chosen from, butylated hydroxytoluene and tromethamine. In some embodiments, the at least one antioxidant is butylated hydroxytoluene. In some embodiments, the at least one antioxidant is tromethamine.

In some embodiments, the at least one antioxidant is present in an amount ranging from 0.05-1% by weight per volume of the coating composition.

At Least One Anti-Proliferative Agent

In some embodiments, the composition further comprises at least one anti-proliferative agent. In some embodiments, when at least one anti-proliferative agent is present in the composition, the composition further comprising at least one anti-proliferative agent is formed by combining a composition comprising at least one anti-proliferative agent and a composition comprising at least one active agent. In some embodiments, the composition comprising at least one active agent is a composition described herein.

In some embodiments, when at least one anti-proliferative agent is present in the composition, the composition further comprising at least one anti-proliferative agent is formed by combining a composition comprising at least one anti-proliferative agent and a composition comprising at least one active agent prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 24 hours prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 6 hours prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 1 hour prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent less than 2 hours prior to use.

In some embodiments, the at least one anti-proliferative agent is chosen from paclitaxel, paclitaxel derivatives, rapamycin, rapamycin derivatives, and pharmaceutically acceptable salts thereof. In some embodiments, the at least one anti-proliferative agent is paclitaxel. In some embodiments, the paclitaxel derivatives are chosen from docetaxel, and cabazitaxel. In some embodiments, the at least one anti-proliferative agent is rapamycin. In some embodiments, the rapamycin derivatives are chosen from everolimus, ridaforolimus, tacrolimus, umirolimus, and zotarolimus.

In some embodiments, when at least one anti-proliferative agent is present in the composition, the ratio of the volume of a composition comprising at least one anti-proliferative agent to the volume of a composition comprising at least one active agent ranges from 0.5:1 to 2.5:1 prior to combination. In some embodiments, when at least one anti-proliferative agent is present in the composition, the ratio of the volume of a composition comprising at least one anti-proliferative agent to the volume of a composition comprising at least one active agent ranges from 0.5:1 to 1.5:1 prior to combination. In some embodiments, when at least one anti-proliferative agent is present in the composition, the ratio of the volume of a composition comprising at least one anti-proliferative agent to the volume of a composition comprising at least one active agent ranges from 0.75:1 to 1.25:1 prior to combination.

In some embodiments, when at least one anti-proliferative agent is present in the composition, the ratio of the volume of a composition comprising at least one anti-proliferative agent to the volume of a composition comprising at least one active agent is 0.5:1 prior to combination. In some embodiments, when at least one anti-proliferative agent is present in the composition, the ratio of the volume of a composition comprising at least one anti-proliferative agent to the volume of a composition comprising at least one active agent is 0.75:1 prior to combination. In some embodiments, when at least one anti-proliferative agent is present in the composition, the ratio of the volume of a composition comprising at least one anti-proliferative agent to the volume of a composition comprising at least one active agent is 1:1 prior to combination. In some embodiments, when at least one anti-proliferative agent is present in the composition, the ratio of the volume of a composition comprising at least one anti-proliferative agent to the volume of a composition comprising at least one active agent is 1.25:1 prior to combination. In some embodiments, when at least one anti-proliferative agent is present in the composition, the ratio of the volume of a composition comprising at least one anti-proliferative agent to the volume of a composition comprising at least one active agent is 1.5:1 prior to combination. In some embodiments, when at least one anti-proliferative agent is present in the composition, the ratio of the volume of a composition comprising at least one anti-proliferative agent to the volume of a composition comprising at least one active agent is 1.75:1 prior to combination. In some embodiments, when at least one anti-proliferative agent is present in the composition, the ratio of the volume of a composition comprising at least one anti-proliferative agent to the volume of a composition comprising at least one active agent is 2:1 prior to combination. In some embodiments, when at least one anti-proliferative agent is present in the composition, the ratio of the volume of a composition comprising at least one anti-proliferative agent to the volume of a composition comprising at least one active agent is 2.25:1 prior to combination. In some embodiments, when at least one anti-proliferative agent is present in the composition, the ratio of the volume of a composition comprising at least one anti-proliferative agent to the volume of a composition comprising at least one active agent is 2.5:1 prior to combination.

In some embodiments, when at least one anti-proliferative agent is present in the composition, the molar ratio of the at least one anti-proliferative agent to the at least one active agent ranges from 0.25:4 to 4:0.25. In some embodiments, when at least one anti-proliferative agent is present in the composition, the molar ratio of the at least one anti-proliferative agent to the at least one active agent ranges from 0.75:2 to 2:0.75. In some embodiments, when at least one anti-proliferative agent is present in the composition, the molar ratio of the at least one anti-proliferative agent to the at least one active agent ranges from 0.75:1.5 to 1.25:1.5. In some embodiments, when at least one anti-proliferative agent is present in the composition, the molar ratio of the at least one anti-proliferative agent to the at least one active agent ranges from 0.9:1.3 to 1.1:1.5. In some embodiments, when at least one anti-proliferative agent is present in the composition, the molar ratio of the at least one anti-proliferative agent to the at least one active agent is 1:1.4.

Composition Properties

Composition pH

In some embodiments, the composition has a pH ranging from 4 to 8. In some embodiments, the composition has a pH ranging from 5 to 7. In some embodiments, the composition has a pH ranging from 5.5 to 6.5.

Without being bound by any theory, in some embodiments, a composition having a pH ranging from 5 to 7 may be suitable for intraarterial or intravenous delivery and/or may result in a composition where the at least one active agent does not degrade after a period of time compared to a composition having a pH outside of that range, as described herein below.

In some embodiments, the composition has a pH of 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7. In some embodiments, the composition has a pH of 5. In some embodiments, the composition has a pH of 5.1. In some embodiments, the composition has a pH of 5.2. In some embodiments, the composition has a pH of 5.3. In some embodiments, the composition has a pH of 5.4. In some embodiments, the composition has a pH of 5.5. In some embodiments, the composition has a pH of 5.6. In some embodiments, the composition has a pH of 5.7. In some embodiments, the composition has a pH of 5.8. In some embodiments, the composition has a pH of 5.9. In some embodiments, the composition has a pH of 6. In some embodiments, the composition has a pH of 6.1. In some embodiments, the composition has a pH of 6.2. In some embodiments, the composition has a pH of 6.3. In some embodiments, the composition has a pH of 6.4. In some embodiments, the composition has a pH of 6.5. In some embodiments, the composition has a pH of 6.6. In some embodiments, the composition has a pH of 6.7. In some embodiments, the composition has a pH of 6.8. In some embodiments, the composition has a pH of 6.9. In some embodiments, the composition has a pH of 7.

Systems and methods consistent with the invention generally relate to apparatus and methods to treat an arteriovenous fistula (AVF). More particularly, systems and methods consistent with the invention relate to the catheters, and catheter systems to deliver drugs to an AVF and promote AVF maturation.

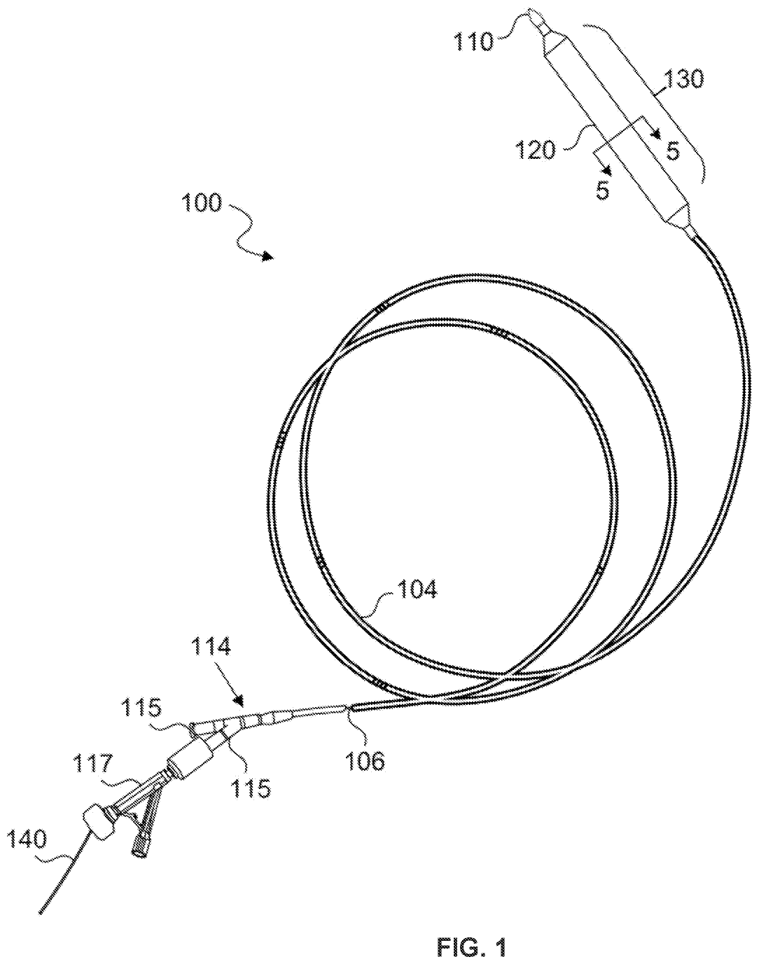

FIG. 1 illustrates an apparatus 100 in accordance with an embodiment of this disclosure. The apparatus 100 having a catheter shaft 104 that extends from a proximal end 106 to a distal tip 110 of the apparatus 100. The apparatus 100 may be configured for longitudinal movement and positioning within a vessel (e.g. blood vessel) of a subject. In some embodiments, the apparatus 100 may be configured for treatment of an area of the vessel. In some embodiments, the apparatus 100 may occlude the vessel, while in other embodiments the apparatus may not occlude the vessel. In some embodiments, the apparatus 100 may be configured for delivery of a drug to an area of the vessel occupied by the apparatus 100 which may form and cast a shape in the vessel, as will be described in more detail below. In other embodiments, the apparatus 100 may be configured for delivery of a light source, a sensor (e.g. a thermocouple), and combinations thereof in the absence of drug delivery or to augment drug delivery.

The length of the catheter shaft 104 may be optimized for canulization of an AVF. For Example, the length of the catheter shaft may be from 20 cm to 40 cm.

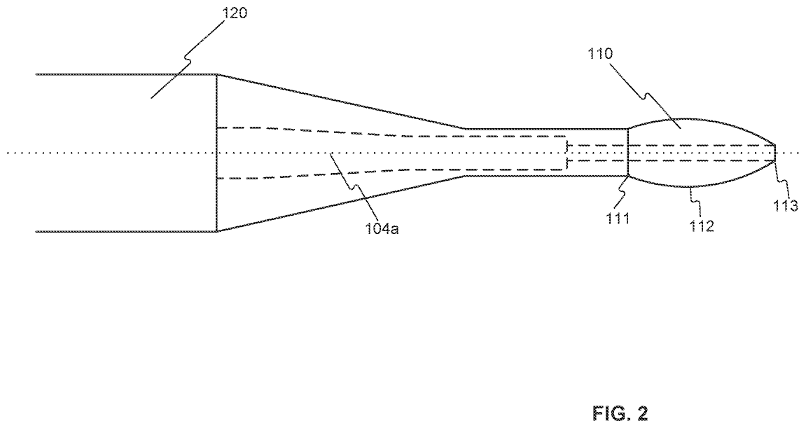

As seen in FIGS. 1-3F, the distal tip may have a variety of shapes. For example, as seen in FIG. 2, the distal tip 110 is olive shaped. As seen in FIG. 2, The olive shaped distal tip 110 may include a proximal portion 111, an intermediate portion 112, and a distal portion 113 with respect to the coated balloon 120, the intermediate portion 112 being disposed between the distal portion 113 and the proximal portion 111. The distal tip 110 having an olive shape may be wider at the intermediate portion 112 than at the distal portion 113 and the proximal portion 111 in a direction perpendicular to an axis 104a of the catheter shaft 104. Additionally, the distal tip 110 having an olive shape may be wider proximal portion 111 than the distal portion 113 in the direction perpendicular to the axis 104a of the catheter shaft 104. The distal tip 110 having an olive shape may advantageously allow for improved vessel dilation during canulization of an AVF, as well as ease introduction of the tip into the vein or artery and ease advancement through the vessel.

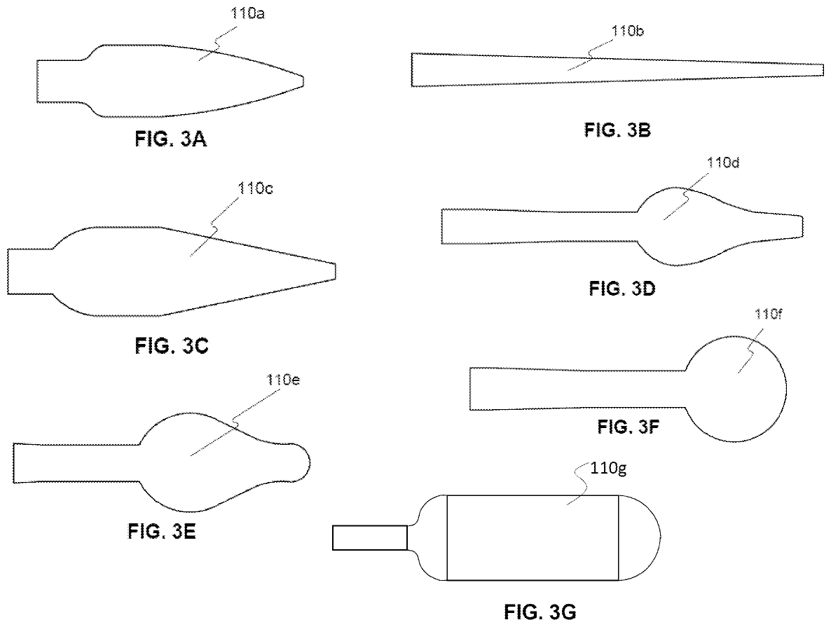

As seen in FIGS. 3A-3G, other shapes for the distal tip 110 may be utilized to achieve effective vessel dilation or ease insertion or advancement during canulization of an AVF. For example, a taper extending from the distal portion 113 of distal tips 110A-D may help to ease insertion of the apparatus into a vein during canulization of an AVF. In other arrangements, a blunt profile of the distal portion 113 of distal tips 110E-G may push against a vessel wall during canulization of an AVF and limit pushability along the vessel. Additionally, bulbous designs of distal tips 110A and 110C-G may help with position tracking of the apparatus in a vessel during canulization of an AVF even if a guidewire is not used. Other distal tip 110 shapes, such as being chamfered or spherical may aid with movement during canulization.

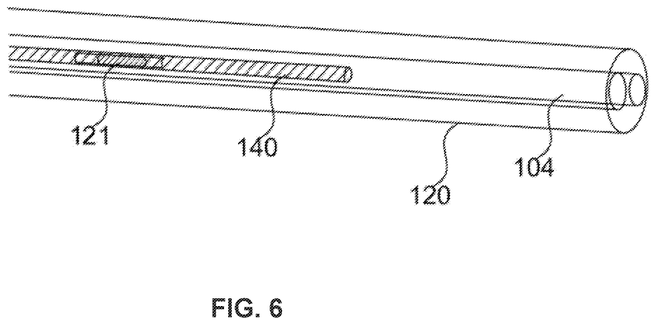

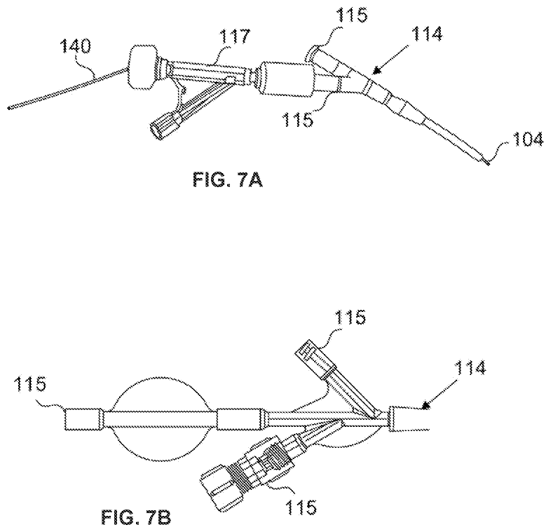

The apparatus 100 may include a proximal end connector 114, shown in more detail at FIGS. 7A and 7B, positioned at the proximal end of the apparatus 100, and the catheter shaft 104 may extend in a distal direction therefrom. The catheter shaft 104 may define one or more lumens that are accessible via a plurality of ports 115 of the proximal end connector 114. The plurality of ports 115 may be configured to engage with external sources desirable to communicate with the plurality of lumens. The ports may engage with external sources via a variety of connection mechanisms, including, but not limited to, syringes, over-molding, quick-disconnect connectors, latched connections, barbed connections, keyed connections, threaded connections, or any other suitable mechanism for connecting one of the plurality of ports to an external source. Non-limiting examples of external sources may include inflation sources (e.g. saline solutions), gaseous sources, treatment sources (e.g. medication, drugs, or any desirable treatment agents discussed further below), light sources (e.g. an integrated light source, a light fiber, a plurality of light-emitting diodes (LEDs)), among others. In some embodiments, apparatus 100 can be used with a guide wire (not shown), via guide wire lumen 164 (see FIG. 8), to assist in guiding the catheter shaft 104 to the target area of the vessel.

FIGS. 1, 2, and 4-6 illustrate the apparatus 100 may include a coated balloon 120 positioned over a distal segment 130 of the catheter shaft 104 proximal to the distal tip 110. In some embodiments, the coated balloon 120 may be proximally offset from the distal tip 110 a distance between 0 mm and 1 mm, 0 mm and 2 mm, 0 mm and 3 mm, 0 mm and 10 mm, or 0 and 50 mm. The coated balloon 120 may take any shape suitable for supporting a wall of a blood vessel or other hollow body structure of the subject when the compliant or semi-compliant balloon is inflated. For example, the coated balloon 120 may expand into a cylindrical shape surrounding the distal segment 130 of the catheter shaft 104. The cylindrical shape may be gradually tapered inward at a proximal end and a distal end of the coated balloon 120, thereby providing a gradually tapered proximal end and distal end of the coated balloon 120 that taper into contact with and become flush with the catheter shaft 104.

Non-limiting examples of shapes the inflated coated balloon 120 may form include a cylindrical shape, football-shaped, spherical, ellipsoidal, or may be selectively deformable in symmetric or asymmetric shapes so as to limit the potential difference in the treated vessel shape and the untreated vessel shape reducing edge effects common between two surfaces of different stiffness as found in metal stents. The force exerted against a vessel interior by coated balloon 120 may be strong enough to scaffold the vessel wall with the apparatus 100 held in a stationary position within the vessel or other hollow body structure. However, the force is not so great as to damage the interior surface of the vessel or other hollow body structure. The coated balloon 120 may be substantially translucent.

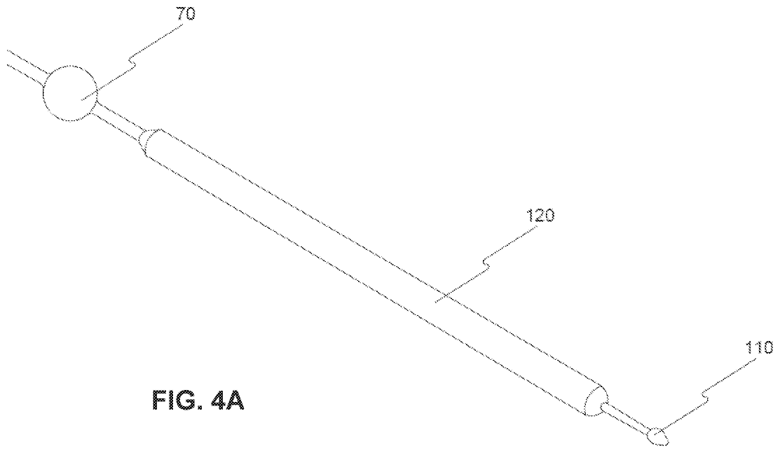

As seen in FIG. 4A, in addition to the coated balloon 120, the apparatus may include a protrusion/proximal bump 70 (e.g., another inflatable balloon). The proximal bump may be utilized a gripping point to aid in the removal of the catheter from a patient after canulization of an AVF.

As seen in FIG. 4B, in addition to the coated balloon 120, the apparatus may include a coated shoulder 72. The coated shoulder provides a surface coated with NVF that, when placed in contact with the cut end of a vessel, e.g. a vein, permits NVF to diffuse into the cut end. Data suggests a high failure rate where the cut vein end is connected to an artery during fistula formation. The treatment of the cut end with NVF can advantageously mitigate this failure mode.

The apparatus 100 may include a plurality of connectors 115 positioned proximally to the proximal end connector 114. For example, the coated balloon 120 may be terminated at the proximal end 106 with a connector capable of receiving an inflation source. In some embodiments, the connector may be a luer configuration. An inflation lumen (discussed in more detail below), may be terminated at the proximal end with a connector capable of receiving a fluid source for clearing the lumen from the proximal termination to outside the distal tip, and in some embodiments may include a luer configuration. The guidewire lumen may also accommodate a guidewire for tracking the catheter apparatus to the desired anatomical location. As discussed in more detail below, the apparatus 100 may also include light fibers that may be terminated at the proximal end with an adaptor capable of connecting with a light source. Each light fiber may terminate with a separate and distinct adaptor or each light fiber may share an adaptor to a light source. The light fibers may be integrated into the apparatus 100 and may be integrated into one of the center lumen, the inflation lumen, and/or as a part of the guidewire itself.

The materials of the apparatus 100 may be biocompatible. The catheter shaft 104 may include material that is extrudable and capable of sustaining lumen integrity. The distal segment 130 of the catheter shaft 104 is substantially translucent to allow light transmission from light fibers. The catheter shaft 104 material is rigid enough to track over a guidewire and soft enough to be atraumatic. The catheter shaft 104 may be made of materials including, but not limited to polymers, natural or synthetic rubber, metal and plastic or combinations thereof, nylon, polyether block amide (PEBA), nylon/PEBA blend, thermoplastic copolyester (TPC), a non-limiting example may be HYTREL@ (available from Dupont de Nemours, Inc. of Wilmington, Delaware), and polyethylene. The shaft materials can be selected so as to maximize column strength to the longitudinal length of the shaft. Further, the shaft materials can be braided, so as to provide sufficient column strength. The shaft materials can also be selected so as to allow the device to move smoothly along a guide wire. The catheter shaft 104 can also be provided with a lubricious coating as well as antimicrobial and antithrombogenic coatings. The shaft materials should be selected so as not to interfere with the efficacy of the agent to be delivered or collected. This interference may take the form of absorbing the agent, adhering to the agent or altering the agent in any way. The catheter shaft 104 of the present disclosure may be between about 2-16 French units (“Fr.” where one French equals % A of a millimeter, or about 0.013 inches). The catheter shafts to be used in coronary arteries may be between about 3-5 Fr. in diameter, and more specifically may be 3 Fr. The catheter shafts to be used in peripheral vessels may be between about 3-8 Fr. in diameter, and more specifically 5 Fr. The catheter shafts to be used in the aorta may be between about 8-16 Fr. in diameter, and more specifically 12 Fr.

The coated balloon 120 may be functionally translucent permitting light, or relevant wavelengths of light, from light fibers to be transmitted substantially beyond the inflated diameter of the coated balloon 120. The coated balloon 120 may be compliant such that the material conforms substantially to a vessel's morphology. The coated balloon 120 material may be elastic, capable of elastically conforming substantially to a vessel's morphology thereby providing optimal drug delivery in a non-dilating and non-traumatic manner. The apparatus 100 may not cause any further trauma (e.g. vessel preparation methods) to the vessel to promote optimal healing.

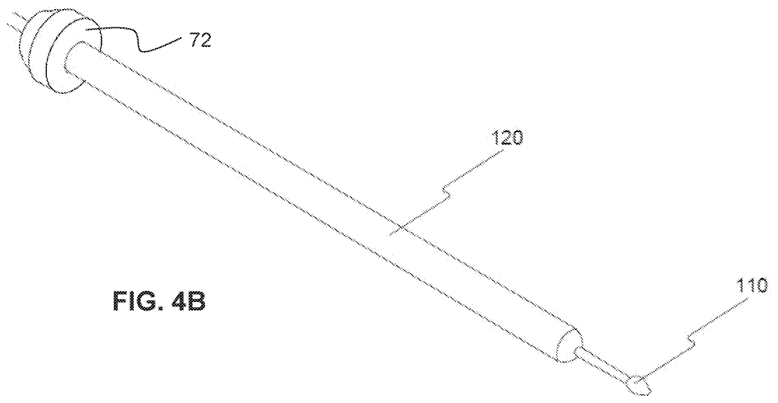

FIG. 5 illustrates the coated balloon 120 that may be coated with at least one drug composition including at least on active compound (e.g., Natural Vascular Scaffolding (NVS) compound), which may be activated by light as discussed further below. The expansion of the coated balloon 120 may shape the treatment area (e.g. vessel) as desired and may provide the drug composition coated on the external surface of the coated balloon 120 to the treatment area.

The at least one active agent may be chosen from the Compound of Formula (I) (and pharmaceutically acceptable salts thereof):

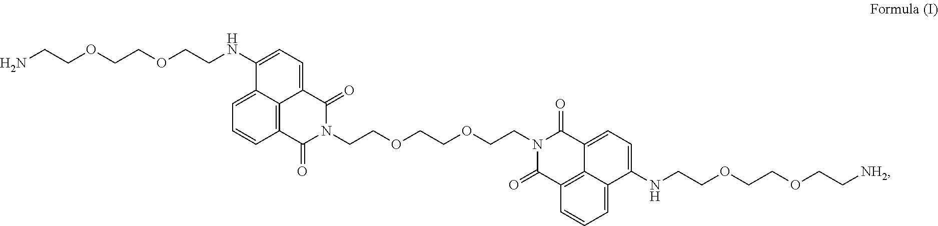 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

In some embodiments, the at least one active agent is administered to the AVF at the time of cannulation. In some embodiments, the methods comprise administering to the AVF a composition comprising at least one active agent chosen from a Compound of Formula (I) and pharmaceutically acceptable salts thereof and illuminating the at least one active agent on said AVF with visible light, such as, for example, 450 nm light.

It has been surprisingly and unpredictably discovered that treatment of an AVF with at least one active agent chosen from the Compound of Formula (t) and pharmaceutically acceptable salts thereof results in a significant increase in AVF open lumen area, without significantly affecting the area of neointimal hyperplasia. Without being bound by theory, it appears the treatment protects the extracellular matrix at the luminal wall from damage by the increased pulsatile pressure. This decreases proliferative signals at the luminal wall leading to increased outward remodeling.

The present disclosures present a novel technology which helps to preserve the natural ECM scaffold using photochemical activation by a small molecule and 450 nm light during the process of vascular dilation by angioplasty balloon. During photoactivation, the ECM fibers are relinked at a position they have been stretched by the balloon inflation. Durable covalent bonds form between amino acids of collagen and elastin fibers, which help retain the enlarged lumen size, but remain flexible to intravascular pressure changes unlike stent implants. This scaffolding effect helps to reduce the sudden hemodynamic impact by the changing blood flow and pressure the vein receives following fistula creation and indirectly contributes to the regulation of subsequent cellular responses.

In some embodiments, provided herein is a composition comprising at least one active agent; at least one tonicity agent; at least one buffer; and at least one vehicle.

In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle. In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle, wherein the composition has a pH of 6. In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle, wherein the composition has a pH of 6, and wherein the at least one active agent does not degrade 156 weeks after preparation of the composition.

In some embodiments, provided herein is a composition comprising at least one active agent chosen from Compound of Formula (I) and pharmaceutically acceptable salts thereof; at least one tonicity agent chosen from potassium chloride and sodium chloride; at least one buffer chosen from potassium phosphate and sodium phosphate; and at least one vehicle comprising water. In some embodiments, the composition has a pH of 6. In some embodiments, provided herein is a composition comprising at least one active agent chosen from Compound of Formula (I) and pharmaceutically acceptable salts thereof; at least one tonicity agent chosen from potassium chloride and sodium chloride; at least one buffer chosen from potassium phosphate and sodium phosphate; and at least one vehicle comprising water, wherein the composition has a pH of 6, and wherein the at least one active agent does not degrade 156 weeks after preparation of the composition.

In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water. In some embodiments, the composition further comprises at least one active agent. In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water, wherein the composition has a pH of 6. In some embodiments, the composition further comprises at least one active agent. In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water, wherein the composition has a pH of 6, and wherein the at least one active agent does not degrade 156 weeks after preparation of the composition. In some embodiments, degradation is determined by LC. In some embodiments, the composition further comprises at least one active agent. In some embodiments, the composition further comprises at least one anti-proliferative agent.

In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, wherein the composition has a pH of 6. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, wherein the composition has a pH of 6, and wherein the at least one active agent does not degrade 156 weeks after preparation of the composition. In some embodiments, degradation is determined by LC. In some embodiments, the composition further comprises at least one active agent. In some embodiments, the composition further comprises at least one anti-proliferative agent.

In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent. In some embodiments, the composition further comprises at least one anti-proliferative agent. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent. In some embodiments, the composition further comprises at least one anti-proliferative agent. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent, wherein the at least one imaging agent comprises a radiographic contrast agent. In some embodiments, the composition further comprises at least one anti-proliferative agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent, wherein the at least one active agent does not degrade 24 hours after preparation of the composition. In some embodiments, degradation is determined by LC. In some embodiments, the composition further comprises at least one active agent. In some embodiments, the composition further comprises at least one anti-proliferative agent.

In some embodiments, the at least one active agent is chosen from a Compound of Formula (I) and pharmaceutically acceptable salts thereof. In some embodiments, the at least one active agent is a diacetate salt of a Compound of Formula (I).

In some embodiments, the at least one active agent is a diacetate salt of a Compound of Formula (I) present in an amount of 0.01% to 1% by weight of the composition. In some embodiments, the at least one active agent is a diacetate salt of a Compound of Formula (I) present in an amount of 0.01% to 1% by weight of the composition and the at least one tonicity agent is chosen from potassium chloride and sodium chloride. In some embodiments, the at least one active agent is a diacetate salt of a Compound of Formula (I) present in an amount of 0.01% to 1% by weight of the composition and the at least one tonicity agent is chosen from potassium chloride and sodium chloride present in an amount ranging from 0.5% to 1.5% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; and the at least one buffer is chosen from sodium phosphate dibasic and potassium phosphate monobasic. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; and the at least one buffer is chosen from sodium phosphate dibasic and potassium phosphate monobasic present in an amount of 0.1% to 0.16% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, wherein the composition has a pH of 6.

In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, wherein the pH of the composition is 6.

In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose in an amount ranging from 0.1% to 0.3% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose in an amount ranging from 0.1% to 0.3% by weight of the composition, wherein the composition has a pH of 6.

The coated balloon 120 may be expandable from a folded or compressed position or orientation to an expanded position or orientation (FIG. 8). In some embodiments, the coated balloon 120 may be in a compressed position, which may be a folded configuration, when the catheter shaft 104 is guided to the target area of the vessel. The coated balloon 120 may undergo a folding and/or wrapping process that wraps the coated balloon 120 around the shaft to reduce the cross-sectional area and to protect the area of the coated balloon 120 under the folds protects the drug from being washed away in the blood stream. The wrapping amount of the coated balloon 120 may be determined by the ratio of the inflated balloon to the wrapped balloon, this ratio may be dictated by the shaft diameter. In some embodiments, a larger wrapping amount may be preferred. As will be discussed in more detail below, advantages of embodiments of the present disclosure provide a smaller catheter shaft 104 diameter, the smaller shaft diameter allows an increase in the amount of the wrapped balloon 120 which will reduce the amount of drug coating that is lost in the bloodstream. Further advantages of embodiments of the disclosure provide for the catheter shaft 104 to have a smaller profile that allows the catheter shaft 104 to be used in smaller vessels and vasculature. Further, the light source and/or light fiber being integrated into the catheter shaft 104 provides for ease of use by a doctor and/or practitioner by eliminating the step of inserting and/or removing a light source or light fiber from the assembly during procedures utilizing the assembly.

The compressed or folded configuration may protect the coated material on the outside surface of the coated balloon 120 when the catheter shaft 104 is guided to a target area of the vessel. When the coated balloon 120 is positioned in the target area, the coated balloon 120 may be inflated into an expanded position, exposing the protected coated material to the treatment site and/or treatment area.

As seen in FIG. 5 the coated balloon 120 may include marker bands 122 positioned at a proximal end and a distal end of the coated balloon 120. The marker bands 122 may allow for precise location tracking of the coated balloon 120 during a procedure such that a user (e.g. a surgeon) may be able to readily locate the coated balloon 120 within an imaging system such as angiography/fluoroscopy. In some embodiments, the marker bands 120 may be radiopaque gold or platinum bands that are integrated into the apparatus 100.

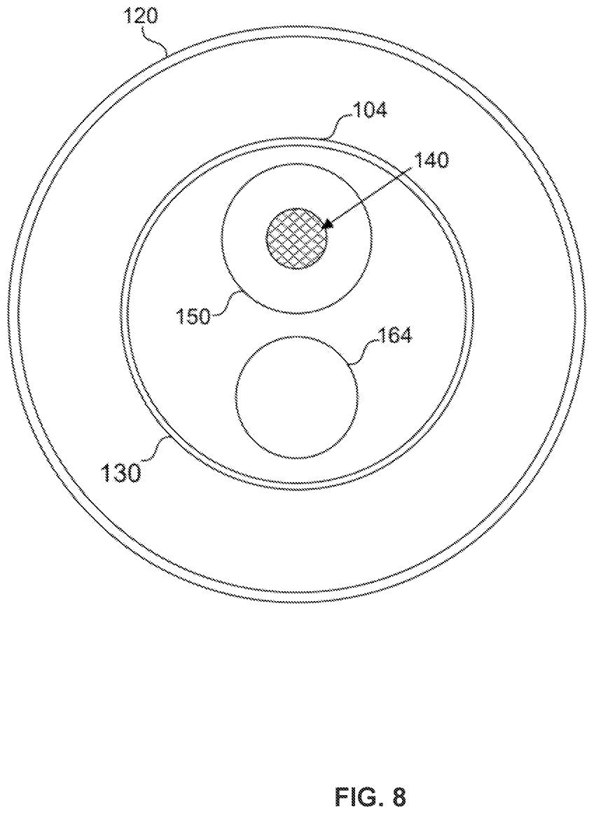

As an alternative to radiographic tracking techniques such as angiography/fluoroscopy (or in addition to), it may be desirable to precisely track a coated balloon 120 via ultrasound. This tracking may be enhanced by the addition of echogenic features to the apparatus 100. For example, the marker bands 122 may be designed to include drill holes (see FIG. 9A) or a roughened surface (see FIG. 9B) that act to disrupt ultrasound waves and increase visibility during ultrasound imaging.

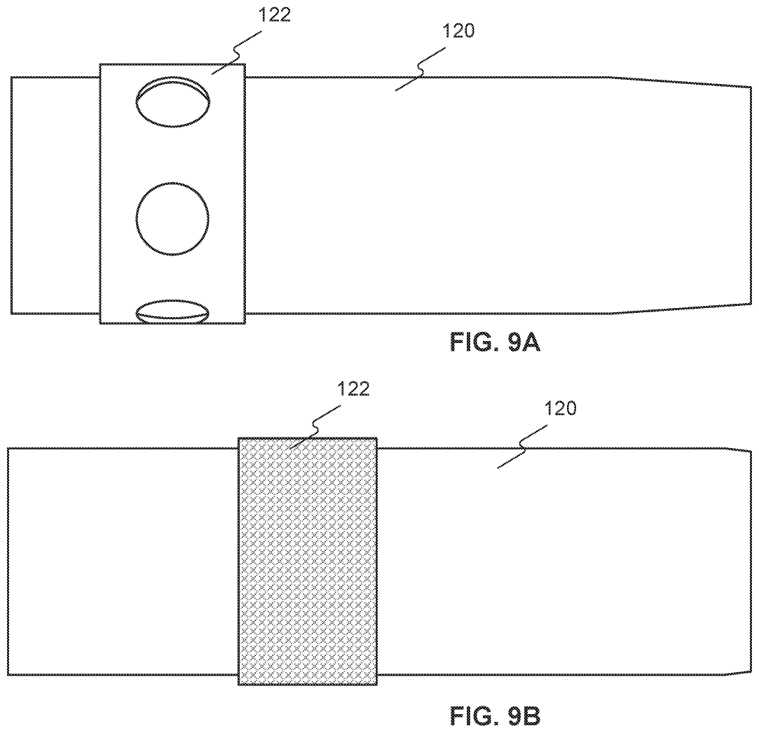

In some arrangements, echogenic features may be added directly the distal tip 110. For example, as seen in FIG. 10A, a roughened or patterned finish may be applied to an outer surface (or not shown inner surface) of the distal tip for increased echogenicity. Such a roughened or patterned finish may be molded directly onto the tip 110, applied via grit blast post-molding, applied via an additional reflow process, or any other suitable method for producing a texture. Alternatively (or in addition), as seen in FIG. 10B, an echogenic rod/tube 50 may be inserted into the distal tip 110. The rod/tube 50 may be, for example, metal with a roughened or patterned surface (e.g., via grit blast, knurling, laser drilling, chemical etching, or any other suitable method for producing a texture).

In some embodiments, the light fiber 140 may be integrated into the apparatus 100. As used herein, the term “integrated” may refer to the light fiber and/or light source being over molded into the apparatus 100 and/or secured within apparatus 100 via adhesive or other securing mechanisms such as a hemostasis valve or other mechanical locking mechanisms, such that the light fiber becomes a non-interchangeable element of the apparatus 100. In some embodiments, the light fiber may be integrated into the apparatus 100 at the time of manufacture. In other embodiments, the light fiber may be integrated into the apparatus 100 in a catheter lab during a clinical preparation process.

The light fiber 140 may be positioned in the catheter shaft 104 and extend through the distal segment 130. The light fiber 140 may transmit light through the distal segment 130 and the coated balloon 120. The light fiber 140 may be connected to the proximal end connector 114 and may have proximal ends that connect to a light fiber activation source via at least one of the plurality of ports 115. In some embodiments, the light fiber 140 may be configured to transmit light at a wavelength of 375 nanometers (nm) to 475 nm, and more specifically 450 nm that transmits through the distal segment 130 and the coated balloon 120. The light fiber 140 may emit light outside of the ultraviolet (UV) range of 10 nm to 400 nm. In some embodiments, the light fiber 140 may be positioned in the light fiber lumen 158, and the light fiber 140 may be covered or shielded along the length of the catheter shaft 104 so that light is only transmitted out of the distal segment 130 and the coated balloon 120.

In some embodiments, the light fiber 140 may be made from plastic core and cladding. The refractive index of the core is high. The refractive index of the cladding is low. A non-limiting example of the core material may be polymethyl methacrylate (PMMA). A non-limiting example of the cladding may be a silicone material. The light source may control the wavelength and supplied power of the light fibers 140. The pattern of the breaks in the cladding of the light fiber ensure uniform power distribution to the vessel wall. Longer lengths have a different pattern than shorter lengths. In an illustrative embodiment, the distal lengths of cladding breaks are matched to the length of the balloons.

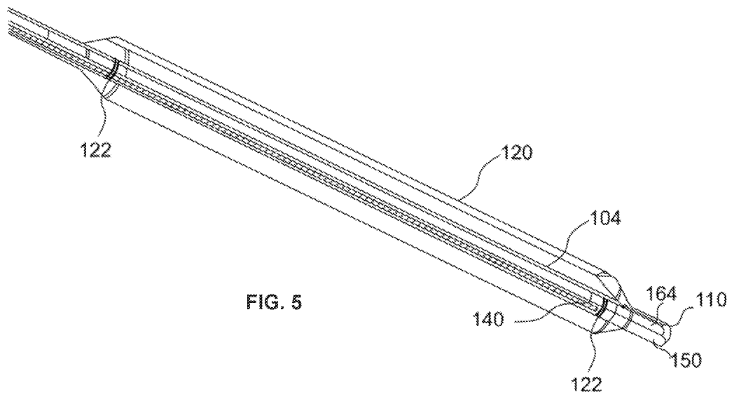

FIG. 6 is a detailed section view of a distal portion of the catheter of FIG. 1. In some embodiments, coated balloon 120 may be connected to inflation lumen via one or more balloon skives 121. The balloon skives 121 may provide fluid communication between the inflation lumen and the coated balloon 120, which may allow the coated balloon 120 to expand outwardly away from catheter shaft 104, as described in further detail below. In some embodiments, the balloon skives 121 may be smaller than light fiber 140 so that the light fiber 140 remains in the inflation lumen and does not enter the coated balloon 120 via balloon skive 121. In some embodiments, any number of balloon skives 121 may be utilized to improve and optimize the flow rate from inflation source into and out of coated balloon 120.

FIG. 7A is a side elevational view of a proximal portion of the catheter, consistent with embodiments of the present disclosure. The apparatus 100 may include a proximal end connector 114 positioned at the proximal end of the apparatus 100, and the catheter shaft 104 may extend in a distal direction therefrom. The catheter shaft 104 may define one or more lumens that are accessible via a plurality of ports 115 of the proximal end connector 114. The plurality of ports 115 may be configured to engage with external sources desirable to communicate with the lumens. The ports may engage with external sources via a variety of connection mechanisms, including, but not limited to, syringes, over-molding, quick-disconnect connectors, latched connections, barbed connections, keyed connections, threaded connections, or any other suitable mechanism for connecting one of the plurality of ports to an external source. Non-limiting examples of external sources may include inflation sources (e.g. saline solutions), gaseous sources, treatment sources (e.g. medication, drugs, or any desirable treatment agents discussed further below), light sources, among others. In some embodiments, apparatus 100 can be used with a guide wire (not shown), via guide wire lumen 164 (see FIG. 8), to assist in guiding the catheter shaft 104 to the target area of the vessel. In some embodiments, the ports 115 may include a hemostasis valve 117 that may be utilized to control the position of light fiber 140 and allow for inflation of coated balloon 120.

FIG. 7B is a side elevational view of another embodiment of proximal portion 114 of apparatus 100, consistent with embodiments of the present disclosure. The catheter shaft 104 may define one or more lumens that are accessible via a plurality of ports 115 of the proximal end connector 114. The plurality of ports 115 may engage with external sources via a variety of connection mechanisms. Non-limiting examples of external sources may include inflation sources (e.g. saline solutions), gaseous sources, treatment sources (e.g. medication, drugs, or any desirable treatment agents discussed further below), light sources, among others. In some embodiments, the ports 115 may include a separate port 115 for controlling the position of light fiber 140, for inflation of coated balloon 120, and for guidewire connection.

FIG. 8 is a cross-sectional view taken along line 5-5 of FIG. 1 showing the lumens within the assembly 100, according to an embodiment of this disclosure. The catheter shaft 104 may have an outside diameter and outside surface 130. The catheter shaft 104 may have an inside configuration of distinct and separate lumens, extending from the proximal end 106 to the distal tip 110.

The coated balloon 120 may be in fluid communication with an inflation lumen 150. The inflation lumen 150 may extend through the catheter shaft 104 and have an input at one of the plurality of ports 115 of the proximal end connector 114. Fluid communication between the coated balloon 120 and the inflation source via the inflation lumen 150 and balloon skives 121 may cause the coated balloon 120 to selectively fill and expand. Light fiber 140 may be integrated into and positioned in inflation lumen 150, and inflation lumen 150 may be designed with a unique lumen geometry to maximize the cross-sectional area of lumen with the light fiber 140 integrated into inflation lumen 150.

A guidewire lumen 164 may also be provided. A guidewire lumen may extend from the proximal end 106 through the distal tip 110. The guidewire lumen 164 may accommodate a guidewire to aid the placement of the apparatus 100 to a desired anatomical position communicating with the proximal end and distal tip. The guidewire may be separate and distinct from the apparatus 100 and extend proximally beyond the proximal end and distally beyond the distal tip of the catheter shaft. The guidewire may remain in the guidewire lumen 104 maintaining anatomical position during the activation of the light fibers.

As shown, the catheter shaft 104 may include a two-lumen extrusion of the inflation lumen 150 and the guidewire lumen 164. In some-embodiments, the guidewire lumen 164 and inflation lumen 150 may be arranged at opposing clockwise positions with respect to each-other in the cross-section of catheter shaft 104. In other embodiments, the light fiber 140 may be integrated into the guidewire lumen 164.

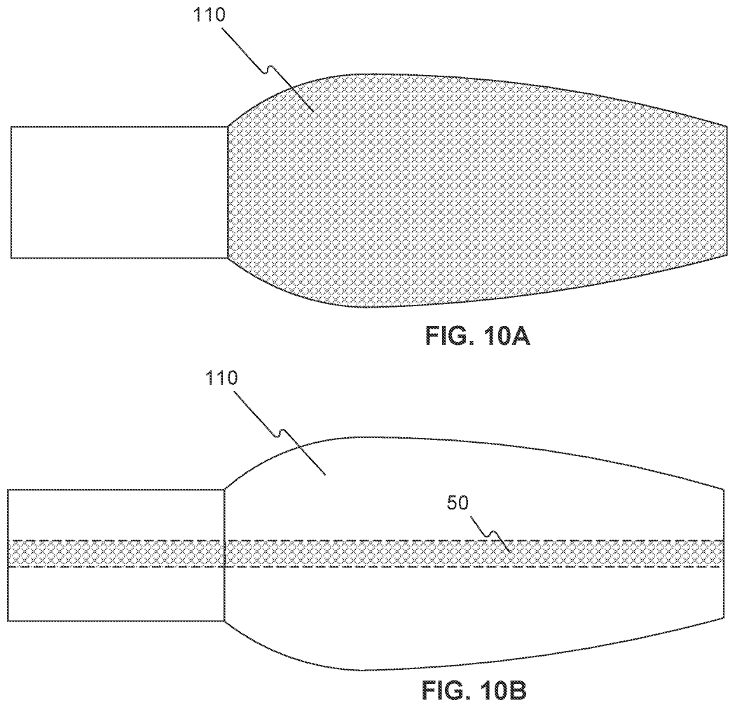

FIG. 11A is a cross-sectional view of an alternative distal end of apparatus 100, which may be an alternative cross-sectional view along the line 5-5 of FIG. 1. The inflation lumen 150 may have a semi-circular or hemi-circular cross-sectional shape and may receive light fiber 140 within the inflation lumen 150. The guidewire lumen 164 may have a circular cross-sectional shape and may be centrally positioned opposite the inflation lumen 150.

FIG. 11B is a cross-sectional view of an alternative distal end of apparatus 100, which may be an alternative cross-sectional view along the line 5-5 of FIG. 1. The inflation lumen 150 may have a semi-circular or hemi-circular cross-sectional shape that extends outward at the edges of the shape to increase the cross-sectional surface area of the inflation lumen and may receive light fiber 140 within the inflation lumen 150. The inflation lumen 150 lumen may form a crescent shape where the inflation lumen 150 forms a curved shape that may be thicker in the middle and tapers to thinner extension sections 151 at each end. The light fiber 140 may be positioned in the thicker middle section of the inflation lumen 150. The guidewire lumen 164 may have a circular cross-sectional shape and may be centrally positioned opposite the inflation lumen 150. In some embodiments, the inflation lumen 150 of FIG. 11B increases the cross-sectional area of the inflation lumen 150 by 50% compared to extrusion of FIG. 11A.

FIG. 11C is a cross-sectional view of an alternative distal end apparatus 100, which may be an alternative cross-sectional view along the line 5-5 of FIG. 1. The inflation lumen 150 shown in FIG. 11C may share a similar cross-sectional profile as inflation lumen 150 shown in FIG. 11B, and inflation lumen 150 of FIG. 11C may further include a support rib 153 that may split inflation lumen 150 into both inflation lumen 150 and a light fiber lumen 158. Light fiber 140 may be integrated in light fiber lumen 158, and the extrusion of catheter shaft 104 may be skived at the proximal hub 114 and at the distal section so that both inflation lumen 150 and light fiber lumen 158 may be used for inflation and deflation of coated balloon 120. As such, inflation lumen 150 and light fiber lumen 158 may be connected.

The catheter shaft 104 embodiments provided in FIGS. 8, 11A, 11B, and 11C allow the catheter shaft 104 and apparatus 100 to have a more compact design by reducing the diameter of the catheter shaft 104. The reduction of the diameter of catheter shaft 104 may be achieved by integrating the light fiber 140 into the inflation lumen 150, which may result in a 50% reduction in the diameter of the catheter shaft 104. The reduction in size and the limited number of lumens may also be advantageous because it may allow for a simpler and streamlined manufacturing process. Furthermore, the apparatus 100 with a reduced diameter may be used in smaller anatomy throughout a subject. For example, apparatus may be used below the knee arteries, in the coronary arteries, among other applications.

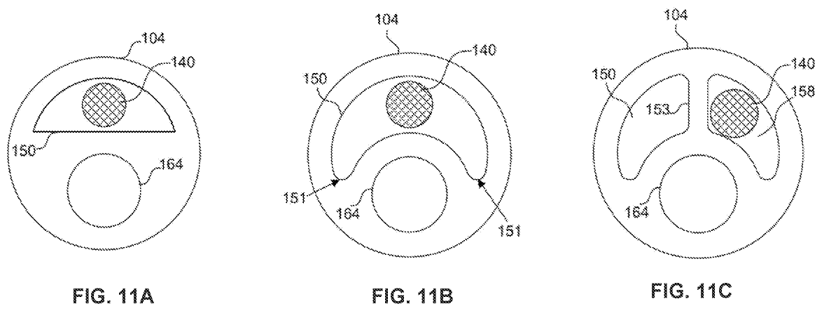

FIGS. 12 to 14B show another embodiment of an apparatus 200 having a coated balloon 220 with a catheter shaft 204 that receives a light fiber 240 that is integrated into the apparatus 200. The coated balloon 220 may have the same or similar features to coated balloon 120 described above. In some embodiments, the apparatus 200 may share many of the same components and features of apparatus 100 described above. The apparatus 200 may include a proximal end connector 214 positioned at the proximal end of the apparatus 200, and the catheter shaft 204 may extend in a distal direction therefrom. The catheter shaft 204 may define one or more lumens that are accessible via a plurality of ports 215 of the proximal end connector 214.

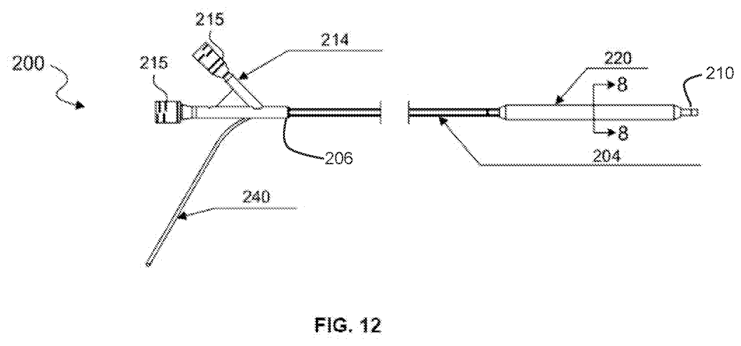

FIG. 13 is a cross-sectional view taken along line 8-8 of FIG. 12 showing the lumens within the assembly 200, according to an embodiment of this disclosure. The catheter shaft 204 may have an outside diameter and outside surface 230. The catheter shaft 204 may have an inside configuration of distinct and separate lumens, extending from the proximal end 206 to the distal tip 210.

Catheter shaft 204 may include two extrusions, an inner extrusion 231 and an outer extrusion 233 that may be heat bonded together using reflow process (e.g. hot air). Inner extrusion 231 may include a notch 235 on an outer surface of inner extrusion 231, the notch 235 may be configured to receive light fiber 240 and/or multiple light fibers. The notch 235 may extend from the proximal end 206 through the distal tip 210. The outer extrusion 233 may be a tube that is heat shrunk onto inner extrusion 231, thereby bonding the catheter shaft 204 together. The materials used for the catheter shaft 204 including the inner extrusion 231 and outer extrusion 233 may be translucent to allow light transmission from light fiber 240.

The coated balloon 220 may be in fluid communication with an inflation lumen 150. The inflation lumen 250 may extend through the catheter shaft 204 and have an input at one of the plurality of ports 215 of the proximal end connector 214. Fluid communication between the coated balloon 220 and the inflation source via the inflation lumen 250 may cause the coated balloon 220 to selectively fill and expand. Skiving for balloon inflation/deflation would be performed through both extrusions.

A guidewire lumen 264 may also be provided. A guidewire lumen may extend from the proximal end 206 through the distal tip 210. The guidewire lumen 264 may accommodate a guidewire to aid the placement of the apparatus 200 to a desired anatomical position communicating with the proximal end and distal tip. The guidewire may be separate and distinct from the apparatus 200 and extend proximally beyond the proximal end and distally beyond the distal tip of the catheter shaft. The guidewire may remain in the guidewire lumen 264 maintaining anatomical position during the activation of the light fiber(s) 240.

As shown, the catheter shaft 104 may include a two-lumen extrusion of the inflation lumen 250 and the guidewire lumen 264. In some-embodiments, the guidewire lumen 264 and inflation lumen 250 may be arranged at opposing clockwise positions with respect to each-other in the cross-section of catheter shaft 104.

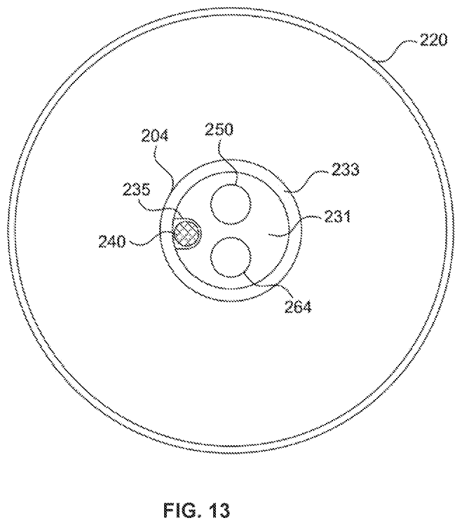

FIG. 14A is a cross-sectional view of the distal end of an embodiment of apparatus 200 showing the inner extrusion 231 and the notch 235 that may be configured to receive one or more light fibers (e.g. light fiber 240). FIG. 14B illustrates an exemplary embodiment having two notches 235 arranged opposite each other on the inner extrusion 231. In some embodiments, FIG. 14B may provide for the use of multiple components within the notches 235. For example, one notch 235 may include a light source and the other notch 235 could include a thermocouple that measures the temperature during activation of the light source. In another example, one notch 235 could include a light source and the other notch 235 could include a sensor that measures light intensity to monitor the output of the light source.

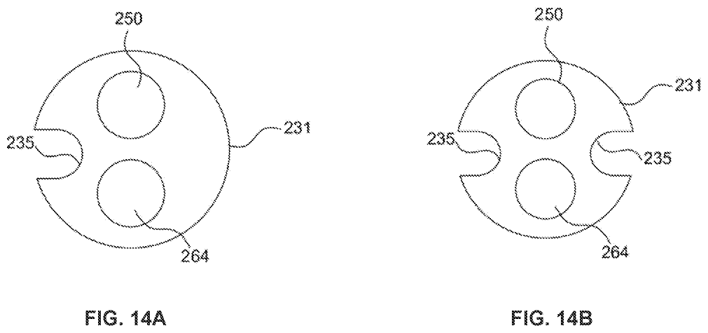

FIG. 15 shows another embodiment of an apparatus 300 having a coated balloon 320 with a catheter shaft 304 that receives a light source 340 that is integrated into the apparatus 300. The coated balloon 320 may have the same or similar features to coated balloon 120, 220 described above. In some embodiments, the apparatus 300 may share many of the same components and features of apparatus 100, 200 described above. The apparatus 300 may include a proximal end connector positioned at the proximal end of the apparatus 300, and the catheter shaft 304 may extend in a distal direction therefrom. The catheter shaft 304 may define one or more lumens that are accessible via a plurality of ports of the proximal end connector.

Light source 340 may be an integrated light source. Non-limiting examples of light source 340 may include a plurality of light-emitting diodes (LEDs) which may be on a strip that is positioned within the distal end of apparatus 300 within the coated balloon 320. The light source 340 may be integrated into catheter shaft 304 at the time of manufacture. Light source 340 may be connected to a power source via a power connection at proximal hub (e.g. proximal hub 114, 214).

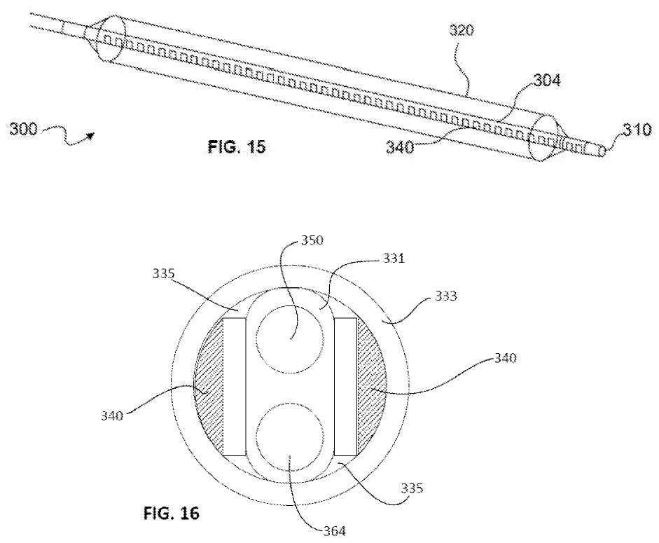

FIG. 16 is a cross-sectional view of FIG. 15, showing the distal end of apparatus 300. As shown, the catheter shaft 304 may include a two-lumen extrusion 331 of the inflation lumen 350 and the guidewire lumen 364. In some-embodiments, the guidewire lumen 364 and inflation lumen 350 may be arranged at opposing clockwise positions with respect to each-other in the cross-section of catheter shaft 304. Catheter shaft 304 may include an inner extrusion 331 and an outer extrusion 333 that may be heat bonded together using reflow process (e.g. hot air).

Inner extrusion 331 may be extruded with light source gaps 335 that provide space to receive one or more light sources (e.g. light source 340) between the inner extrusion 331 and outer extrusion 333. The light source gaps 335 may extend from the proximal end through the distal tip 310. The outer extrusion 333 may be a tube that is heat shrunk onto inner extrusion 331, thereby bonding the catheter shaft 304 together. The materials used for the catheter shaft 304 including the inner extrusion 331 and outer extrusion 333 may be translucent to allow light transmission from light source 340.

Some embodiments of the present disclosure provide a manufacturing method for manufacturing the apparatuses 100, 200, 300 disclosed herein. The manufacturing method may include extruding the inner extrusion (e.g. 231, 331), extruding the outer extrusion (e.g. 233, 333), inserting the light source (e.g. light fiber 140, 240 and/or light source 340) into at least one of the inflation lumen 150, the inner extrusion 231 at notch 235, and the light source gaps 335. The method may further include placing the outer extrusion (e.g. 233, 333) around inner extrusion (e.g. 231, 331) and placing mandrels in the inflation and guidewire lumens to prevent the lumens from collapsing during manufacture. The method may further include applying heat to the outer extrusion to shrink the outer extrusion onto the inner extrusion to bond the extrusions together. The method may further include skiving into balloon inflation lumen (e.g. inflation lumen 150, 250, 350) for balloon inflation/deflation though the outer extrusion and inner extrusion at desired positions along the catheter shaft. The method may further include connecting the proximal end connector to the catheter shaft.

Now that the components of each apparatus 100, 200, 300 have been described in detail, the methods associated with the apparatuses 100, 200, 300 can be appreciated. The target area for a delivery of drug source may be a vessel of the cardiovascular system. In some embodiments, the target area may be first prepared by beginning the creation of an AVF. For example, a Cimino fistula is a surgically created fistula between an artery and vein of the arm (e.g., cephalic vein and radial artery). After insertion of the apparatus 100, 200, 300 to the target area, the functional pressure of the coated balloon 120, 220, 320 is sufficient to prop open the vessel during drug functionalization. The coated balloon 120, 220, 320 may be inflated into contact with the vessel wall in order to uniformly deliver the coated drug to the vessel wall. While in this vessel supported position, a light source may be supplied to the light fibers 140, 240 and/or light source 340 in the catheter shaft 104, 204, 304 for transmittance through the catheter shaft 104, 204, 304, through the coated balloon 120, 220, 320 and into the vessel wall.

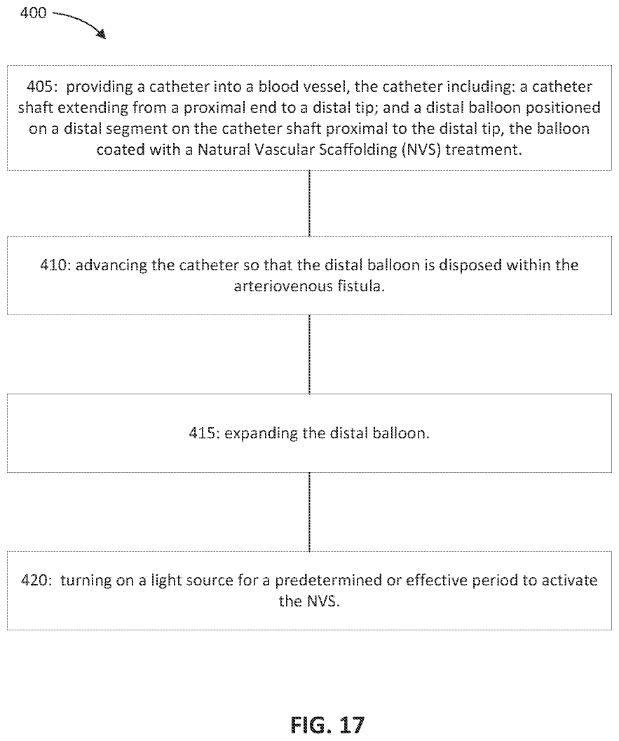

An embodiment of this disclosure provides an exemplary method 400 of tissue restoration in a blood vessel of a subject is depicted in FIG. 17. The method may include providing 405 an apparatus (e.g. apparatus 100, 200, 300) and preparing the apparatus for a clinical procedure, which may include sterilizing the apparatus and connecting the light fiber to the light source and/or for providing power to the light source. The method may further include advancing 410 the apparatus to the treatment site with or without a guidewire using angiography or ultrasound imaging for visualization and aligning the marker bands with the desired treatment site. Subsequently, the balloon may be inflated 415 to a desired pressure based on a sizing chart for the treatment area (e.g. based on the diameter of the treatment vessel) and maintain the inflation of the balloon a predetermined amount of time (e.g. one to three minutes), allowing the drug to transfer into the wall of the vessel.

The method may further include, while the balloon remains inflated, turning on the light source for a predetermined or effective period 420 (e.g. one to three minutes), transmitting light down the light fiber and/or light source which may be integrated into the catheter shaft and allowing the light to activate the drug that has been transported into the vessel. Once complete, the balloon may be deflated and removed. With the integrated light fiber and/or light source (e.g. 140, 240, 340), the light fiber and/or light source does not need to be inserted and/or removed as a process step.

Additionally, therapeutic agents useful with the device of the present disclosure include any one of or a combination of several agents which are gas, liquid, suspensions, emulsions, or solids, which may be delivered or collected from the vessel for therapeutic or diagnostic purposes. Therapeutic agents may include biologically active substances, or substances capable of eliciting a biological response, including, but not limited to endogenous substances (growth factors or cytokines, including, but not limited to basic fibroblast growth factor, acidic fibroblast growth factor, vascular endothelial growth factor, angiogenic factors, microRNA), viral vectors, DNA capable of expressing proteins, sustained release polymers, and unmodified or modified cells. Therapeutic agents may include angiogenic agents which induce the formation of new blood vessels. Therapeutic agents may also include anti-stenosis or anti-restenosis agents which are used to treat the narrowing of blood vessel walls. Therapeutic agents may include light-activated agents such as light-activated anti-stenosis or light-activated anti-restenosis agents that may be used to treat the narrowing of blood vessel walls.

Accordingly, apparatuses 100, 200, 300 are multifunctional, providing drug delivery control in open and closed positions, and propping open a vessel wall forming a shape during drug functionalizing with a light source of a specific wavelength outside of the ultraviolet (UV) range (10 nm to 400 nm).

Accordingly, the apparatus and methods described herein provide the delivery of NVS to a treatment area (e.g. a vessel) and provide restoration to that treatment area using the apparatus or according to the methods described above. The apparatus and method described above provide concurrently treating the vessel with one or more drugs (e.g. with Paclitaxel and NVS) with minimal loss to other vessels, scaffolding and casting the vessel, and light activation of the one or more drugs delivered to the treatment area. These advantages can be accomplished utilizing the apparatus and methods described herein.

The foregoing description has been presented for purposes of illustration. It is not exhaustive and does not limit the invention to the precise forms or embodiments disclosed. Modifications and adaptations of the invention will be apparent to those skilled in the art from consideration of the specification and practice of the disclosed embodiments of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An apparatus comprising
a catheter shaft extending from a proximal end to a distal tip; and
a distal balloon positioned on a distal segment of the catheter shaft proximal to the distal tip, the distal balloon coated with a Natural Vascular Scaffolding (NVS) treatment;
wherein the NVS treatment is configured to enhance maturation of an arteriovenous fistula.

2. The apparatus of claim 1:
wherein the distal tip includes a distal portion, a proximal portion and an intermediate portion, the intermediate portion being disposed between the distal portion and the proximal portion;
wherein the distal tip, in a direction perpendicular to an axis of the catheter shaft, is wider at the intermediate portion than at the distal portion and the proximal portion.

3. The apparatus of claim 2, wherein the distal tip, in the direction perpendicular to the axis of the catheter shaft, is wider at the proximal portion than at the distal portion.

4. The apparatus of claim 2, wherein the distal tip is olive shaped.

5. The apparatus of claim 2, wherein the distal tip is tapered from the intermediate portion to the distal portion.

6. The apparatus of claim 1, wherein the catheter shaft includes echogenic features configured to enhance visibility of the distal tip when viewed with ultrasound.

7. The apparatus of claim 6, wherein the echogenic features include a roughened and/or patterned finish to an outer surface of the distal tip.

8. The apparatus of claim 6, wherein the echogenic features include a roughened and/or patterned finish to an outer surface a rod or a tube disposed within the distal tip.

9. The apparatus of claim 6, wherein the echogenic features include a roughened and/or patterned finish to an inner surface of the distal tip.

10. The apparatus of claim 6, wherein the echogenic features include metal wire braiding and/or metal coiling.

11. The apparatus of claim 1, wherein the catheter shaft includes at least one radiopaque marker band configured to enhance visibility under fluoroscopy.

12. The apparatus of claim 11, wherein the radiopaque marker band includes at least one of radial holes, a roughened surface, and a patterned surface configured to enhance visibility of the distal tip when viewed with ultrasound.

13. The apparatus of claim 1, wherein the catheter shaft includes a protrusion proximal to the distal balloon and configured to aid in removal of the device from a patient.

14. The apparatus of claim 13, wherein the protrusion is a proximal inflatable balloon.

15. A method for maturing an arteriovenous fistula comprising:
providing a catheter into a blood vessel, the catheter including:
a catheter shaft extending from a proximal end to a distal tip; and
a distal balloon positioned on a distal segment of the catheter shaft proximal to the distal tip, the distal balloon coated with a Natural Vascular Scaffolding (NVS) treatment;
advancing the catheter so that the distal balloon is disposed within the arteriovenous fistula; and
expanding the distal balloon.

16. The method of claim 15:
wherein the distal tip includes a distal portion, a proximal portion and an intermediate portion, the intermediate portion being disposed between the distal portion and the proximal portion;
wherein the distal tip, in a direction perpendicular to an axis of the catheter shaft, is wider at the intermediate portion than at the distal portion and the proximal portion.

17. The method of claim 16, wherein the distal tip, in the direction perpendicular to the axis of the catheter shaft, is wider at the proximal portion than at the distal portion.

18. The method of claim 16, wherein the distal tip is olive shaped.

19. The method of claim 16, wherein the distal tip is tapered from the intermediate portion to the distal portion.

20. The method of claim 15, wherein the catheter shaft includes echogenic features configured to enhance visibility of the distal tip when viewed with ultrasound.

21. The method of claim 20, wherein the echogenic features include a roughened and/or patterned finish to an outer surface of the distal tip.

22. The method of claim 20, wherein the echogenic features include a roughened and/or patterned finish to an outer surface of a rod or a tube disposed within of the distal tip.

23. The method of claim 20, wherein the echogenic features include a roughened and/or patterned finish to an inner surface of the distal tip.

24. The method of claim 20, wherein the echogenic features include metal wire braiding and/or metal coiling.

25. The method of claim 15, wherein the catheter shaft includes at least one radiopaque marker band configured to enhance visibility under fluoroscopy.

26. The method of claim 25, wherein the radiopaque marker band includes at least one of radial holes, a roughened surface, and a patterned surface configured to enhance visibility of the distal tip when viewed with ultrasound.

27. The apparatus of claim 15, wherein the catheter shaft includes a protrusion proximal to the distal balloon and configured to aid in removal of the device from a patient.

28. The apparatus of claim 27, wherein the protrusion is a proximal inflatable balloon.
